SEQUENCE LISTING (11 sequences)

SEQ ID NO: 1 - protein, 31 aa, Homo sapiens
EAEDLQVGQVELGGGPGAGSLQPLALEGSLQ

SEQ ID NO: 2 - protein, 30 aa, Homo sapiens
HAEGTFTSDVSSYLEGQAAKEFIAWLVKGR

SEQ ID NO: 3 - protein, 244 aa, Homo sapiens
MLLLGAVLLLLALPGHDQETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 4 - DNA, 4578 bp, Homo sapiens
aggctgttgaggctgggccatctcctcctcacttccattctgactgcagtctgtggttctgattccataccagaggggctcaggatgctgttgctgggagctgttctactgctattagctctgcccggtcatgaccaggaaaccacgactcaagggcccggagtcctgcttcccctgcccaagggggcctgcacaggttggatggcgggcatcccagggcatccgggccataatggggccccaggccgtgatggcagagatggcacccctggtgagaagggtgagaaaggagatccaggtcttattggtcctaagggagacatcggtgaaaccggagtacccggggctgaaggtccccgaggctttccgggaatccaaggcaggaaaggagaacctggagaaggtgcctatgtataccgctcagcattcagtgtgggattggagacttacgttactatccccaacatgcccattcgctttaccaagatcttctacaatcagcaaaaccactatgatggctccactggtaaattccactgcaacattcctgggctgtactactttgcctaccacatcacagtctatatgaaggatgtgaaggtcagcctcttcaagaaggacaaggctatgctcttcacctatgatcagtaccaggaaaataatgtggaccaggcctccggctctgtgctcctgcatctggaggtgggcgaccaagtctggctccaggtgtatggggaaggagagcgtaatggactctatgctgataatgacaatgactccaccttcacaggctttcttctctaccatgacaccaactgatcaccactaactcagagcctcctccaggccaaacagccccaaagtcaattaaaggctttcagtacggttaggaagttgattattatttagttggaggcctttagatattattcattcatttactcattcatttattcattcattcatcgagtaactttaaaaaaatcatatgctatgttcccagtcctggggagcttcacaaacatgaccagataactgactagaaagaagtagttgacagtgctattttgtgcccactgtctctcctgatgctcatatcaatcctataaggcacagggaacaagcattctcctgtttttacagattgtatcctgaggctgagagagttaagtgaatgtctaaggtcacacagtattaagtgacagtgctagaaatcaaacccagagctgtggactttgttcactagactgtgcccttttatagaggtacatgttctctttggagtgttggtaggtgtctgtttcccacctcacctgagagccattgaatttgccttcctcatgaattaaaacctcccccaagcagagcttcctcagagaaagtggttctatgatgacgtcctgtcttggaaggactactactcaatggcccctgcactactctacttcctcttacctatgtcccttctcatgcctttccctccaacggggaaagccaactccatctctaagtgccgaactcatccctgttcctcaaggccacctggccaggagcttctctgatgtgatatccactttttttttttttgagatggagtctcactctgtcacccaggctggagtacagtgacacgacctcggctcactgcagcctccttctcctgggtccaagcaattattgtgcctcagcctcccgagtagctgagacttcaggtgcattccaccacacatggctaatttttgtatttttagtagaaatggggtttcgtcatgttggccaggctggtctcgaactcctggcctaggtgatccacccgcctcgacctcccaaagtgctgggattacaggcatgagccaccatgcccagtcgatatctcactttttattttgccatggatgagagtcctgggtgtgaggaacacctcccaccaggctagaggcaactgcccaggaaggactgtgcttccgtcacctctaaatcccttgcagatccttgataaatgcctcatgaagaccaatctcttgaatcccatatctacccagaattaactccattccagtctctgcatgtaatcagttttatccacagaaacattttcattttaggaaatccctggttttaagtatcaatccttgttcagctggacaatatgaatcttttccactgaagttagggatgactgtgattttcagaacacgtccagaatttttcatcaagaaggtagcttgagcctgaaatgcaaaacccatggaggaattctgaagccattgtctccttgagtaccaacagggtcagggaagactgggcctcctgaatttattattgttctttaagaattacaggttgaggtagttgatggtggtaaacattctctcaggagacaataactccagtgatgttcttcaaagattttagcaaaaacagagtaaatagcattctctatcaatatataaatttaaaaaactatctttttgcttacagttttaaattctgaacaattctctcttatatgtgtattgctaatcattaaggtattattttttccacatataaagctttgtctttttgttgttgttgttgtttttaagatggagtttccctctgttgccaggctagagtgcagtggcatgatctcggcttactgcaacctttgcctcccaggttcaagcgattcttctgcctcagcctcccgagtagctgggaccacaggtgcctaccaccatgccaggctaatttttgtatttttagtaaagacagggtttcaccatattggccaggctggtctcgaactcctgaccttgtgatctgcccgcctccatttttgttgttattttttgagaaagatagatatgaggtttagagagggatgaagaggtgagagtaagccttgtgttagtcagaactctgtgttgtgaatgtcattcacaacagaaaacccaaaatattatgcaaactactgtaagcaagaaaaataaaggaaaaatggaaacatttattcctttgcataatagaaattaccagagttgttctgtctttagataaggtttgaaccaaagctcaaaacaatcaagacccttttctgtatgtccttctgttctgccttccgcagtgtaggctttaccctcaggtgctacacagtatagttctagggtttccctcccgatatcaaaaagactgtggcctgcccagctctcgtatccccaagccacaccatctggctaaatggacatcatgttttctggtgatgcccaaagaggagagaggaagctctctttcccagatgccccagcaagtgtaaccttgcatctcattgctctggctgagttgtgtgcctgtttctgaccaatcactgagtcaggaggatgaaatattcatattgacttaattgcagcttaagttaggggtatgtagaggtattttccctaaagcaaaattgggacactgttatcagaaataggagagtggatgatagatgcaaaataatacctgtccacaacaaactcttaatgctgtgtttgagctttcatgagtttcccagagagacatagctggaaaattcctattgattttctctaaaatttcaacaagtagctaaagtctggctatgctcacagtctcacatctggttggggtgggctccttacagaacacgctttcacagttaccctaaactctctggggcagggttattcctttgtggaaccagaggcacagagagagtcaactgaggccaaaagaggcctgagagaaactgaggtcaagatttcaggattaatggtcctgtgatgctttgaagtacaattgtggatttgtccaattctctttagttctgtcagcttttgcttcatatattttagcgctctattattagatatatacatgtttagtattatgtcttattggtgcatttactctcttatcattatgtaatgtccttctttatctgtgataattttctgtgttctgaagtctactttgtctaaaaataacatacgcactcaacttccttttctttcttccttcctttctttcttccttcctttctttctctctctctctctttccttccttccttcctccttttctttctctctctctctctctctctttttttgacagactctcgttctgtggccctggctggagttcagtggtgtgatcttggctcactgctacctctaccatgagcaattctcctgcctcagcctcccaagtagctggaactacaggctcatgccactgcgcccagctaatttttgtatttttcgtagagacggggtttcaccacattcgtcaggttggtttcaaactcctgactttgtgatccacccgcctcggcctcccaaagtgctgggattacaggcatgagccatcacacctggtcaactttcttttgattagtgtttttgtggtatatctttttccatcatgttactttaaatatatctatattattgtatttaaaatgtgtttcttacagactgcatgtagttgggtataatttttatccagtctaaaaatatctgtcttttaattggtgtttagacaatttatatttaataaaattgttgaatttaa

SEQ ID NO: 5 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 6 - DNA, 469 bp, Homo sapiens
agccctccaggacaggctgcatcagaagaggccatcaagcagatcactgtccttctgccatggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaactagacgcagcccgcaggcagccccacacccgccgcctcctgcaccgagagagatggaataaagcccttgaaccagcaaaa

SEQ ID NO: 7 - protein, 224 aa, Homo sapiens
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 8 - DNA, 2024 bp, Homo sapiens
aaggcaagagatctaggacttctagcccctgaactttcagccgaatacatcttttccaaaggagtgaattcaggcccttgtatcactggcagcaggacgtgaccatggagaagctgttgtgtttcttggtcttgaccagcctctctcatgcttttggccagacagacatgtcgaggaaggcttttgtgtttcccaaagagtcggatacttcctatgtatccctcaaagcaccgttaacgaagcctctcaaagccttcactgtgtgcctccacttctacacggaactgtcctcgacccgtgggtacagtattttctcgtatgccaccaagagacaagacaatgagattctcatattttggtctaaggatataggatacagttttacagtgggtgggtctgaaatattattcgaggttcctgaagtcacagtagctccagtacacatttgtacaagctgggagtccgcctcagggatcgtggagttctgggtagatgggaagcccagggtgaggaagagtctgaagaagggatacactgtgggggcagaagcaagcatcatcttggggcaggagcaggattccttcggtgggaactttgaaggaagccagtccctggtgggagacattggaaatgtgaacatgtgggactttgtgctgtcaccagatgagattaacaccatctatcttggcgggcccttcagtcctaatgtcctgaactggcgggcactgaagtatgaagtgcaaggcgaagtgttcaccaaaccccagctgtggccctgaggcccagctgtgggtcctgaaggtacctcccggttttttacaccgcatgggccccacgtctctgtctctggtacctcccgcttttttacactgcatggttcccacgtctctgtctctgggcctttgttcccctatatgcattgcaggcctgctccaccctcctcagcgcctgagaatggaggtaaagtgtctggtctgggagctcgttaactatgctgggaaacggtccaaaagaatcagaatttgaggtgttttgttttcatttttatttcaagttggacagatcttggagataatttcttacctcacatagatgagaaaactaacacccagaaaggagaaatgatgttataaaaaactcataaggcaagagctgagaaggaagcgctgatcttctatttaattccccacccatgacccccagaaagcaggagggcattgcccacattcacagggctcttcagtctcagaatcaggacactggccaggtgtctggtttgggtccagagtgctcatcatcatgtcatagaactgctgggcccaggtctcctgaaatgggaagcccagcaataccacgcagtccctccactttctcaaagcacactggaaaggccattagaattgccccagcagagcagatctgctttttttccagagcaaaatgaagcactaggtataaatatgttgttactgccaagaacttaaatgactggtttttgtttgcttgcagtgctttcttaattttatggctcttctgggaaactcctccccttttccacacgaaccttgtggggctgtgaattctttcttcatccccgcattcccaatatacccaggccacaagagtggacgtgaaccacagggtgtcctgtcagaggagcccatctcccatctccccagctccctatctggaggatagttggatagttacgtgttcctagcaggaccaactacagtcttcccaaggattgagttatggactttgggagtgagacatcttcttgctgctggatttccaagctgagaggacgtgaacctgggaccaccagtagccatcttgtttgccacatggagagagactgtgaggacagaagccaaactggaagtggaggagccaagggattgacaaacaacagagccttgaccacgtggagtctctgaatcagccttgtctggaaccagatctacacctggactgcccaggtctataagccaataaagcccctgtttacttgaaaaaaaaaa

SEQ ID NO: 9 - protein, 180 aa, Homo sapiens
MKSIYFVAGLFVMLVQGSWQRSLQDTEEKSRSFSASQADPLSDPDQMNEDKRHSQGTFTSDYSKYLDSRRAQDFVQWLMNTKRNRNNIAKRHDEFERHAEGTFTSDVSSYLEGQAAKEFIAWLVKGRGRRDFPEEVAIVEELGRRHADGSFSDEMNTILDNLAARDFINWLIQTKITDRK

SEQ ID NO: 10 - DNA, 1298 bp, Homo sapiens
gcatagaatgcagatgagcaaagtgagtgggagagggaagtcatttgtaacaaaaactcattatttacagatgagaaatttatattgtcagcgtaatatctgtgaggctaaacagagctggagagtatataaaagcagtgcgccttggtgcagaagtacagagcttaggacacagagcacatcaaaagttcccaaagagggcttgctctctcttcacctgctctgttctacagcacactaccagaagacagcagaaatgaaaagcatttactttgtggctggattatttgtaatgctggtacaaggcagctggcaacgttcccttcaagacacagaggagaaatccagatcattctcagcttcccaggcagacccactcagtgatcctgatcagatgaacgaggacaagcgccattcacagggcacattcaccagtgactacagcaagtatctggactccaggcgtgcccaagattttgtgcagtggttgatgaataccaagaggaacaggaataacattgccaaacgtcacgatgaatttgagagacatgctgaagggacctttaccagtgatgtaagttcttatttggaaggccaagctgccaaggaattcattgcttggctggtgaaaggccgaggaaggcgagatttcccagaagaggtcgccattgttgaagaacttggccgcagacatgctgatggttctttctctgatgagatgaacaccattcttgataatcttgccgccagggactttataaactggttgattcagaccaaaatcactgacaggaaataactatatcactattcaagatcatcttcacaacatcacctgctagccacgtgggatgtttgaaatgttaagtcctgtaaatttaagaggtgtattctgaggccacattgctttgcatgccaataaataaattttcttttagtgttgtgtagccaaaaattacaaatggaataaagttttatcaaaatattgctaaaatatcagctttaaaatatgaaagtgctagattctgttattttcttcttattttggatgaagtaccccaacctgtttacatttagcgataaaattatttttctatgatataatttgtaaatgtaaattattccgatctgacatatctgcattataataataggagaatagaagaactggtagccacagtggtgaaattggaaagagaactttcttcctgaaacctttgtcttaaaaatactcagctttcaatgtatcaaagatacaattaaataaaattttcaagcttctttaccaaaaaaaaaaa

SEQ ID NO: 11 - DNA, 38805 bp, Homo sapiens
catgcactgagctctctccctgtctgtgtagagtgttatacttgggaatataaaggaagtgaccaaatcagtgcttgggaagtagatttggcttctttgtttctcacaggacaatctcagccacccctagttagcaccattaactcctcctgagctctgataaaagaatcaaaatttctcaataatttcatccaaaattattaatgaaaataagaattattttgatggctctaacagtgacatttatatcatctgttttatctggagcattctataataagtttatattaagcaaatcaataaaaacctcttacaaaagtatcatcggatactttcctgaacattaaggagaaatctatagaactgaatgaatgagaaccaacaagtaaatatatgtgatcattgtaaccattgttggtgtggggcatttgtcagaactccaatgtgattattaacataggtgagaattaatccactgtgactttgcccattgcttagaaagaacattcatagtttaattatgccttttttgaccaagcacagtggctcatgcctgtaatcccagcactttgggaggccgaggtgggtggatcacctgaggtcaggagttcgagaccagcctgaccaacatggtgaaaccccatctctactaaaaatacaaaaattagctaggtgtggtggtatgcacctataatctcagctacccaggaggctgaggcaggagaatcacttgaacctggaggcagaggttgcagtgagccgagacgcaccattacactccagcctgggtgacagagtgagattccatctcaaaaaaaaaaaaaaaaaattatgcctttttgaagcacatacattttataacatacaactgaatcccttattatattattagttttgatttaatgttttcaaaccatctcccctgatatttctgggagatgggaaacatgttttcttacacctcttgcattccattctcaactcccaactgtcttactgcaatgaacacttaataagaaacagtcaattggtcaattgattgggcaacaggctaaacacactcattccttgtctgttcccacttctttctttactttcccttcctgagtaacttatcctaaagtcattaggtgggtggcagccagatggtggccacacattaaggtagaaaagagagtgtcatgatggttccaagtcagagacctagtagggtgaggatcaagtaggtgttcacgtggagaaacagcccggcctgtgtgtgggagtccaagcaagcagagaaaatgtcgacacagaggggtggcctgaaaaagcagccagagcctaaacagggcatggagaacatatttagggcatgaggtgaggagggcatccatgagtgggaagggatgggtgaggtttcactacataaaggggattgatgaaataagtaaataaagtatactggaagccaggtgtgtcacttttgcagaaaagagtcatggattcagaaagggagaaaactagcaggaatcctatgaaattagattaaaatggatgtatccatgtatattcatacccttctagatagataaatggttagataggtgataaaaagataacaagaggacaagataattagatagacataaatgtatgtatgtgtttgtgtgtgtgtacaaaaaaacatatactccctacttctctccactgatagggctaggtaacaatggcatttcaatagcaatgagcacacttagtggccagatcttggcttattaataccattttccactgaaaggaaccagagctttttagagaaatggctgattccagggccaggattaagaatgttcaagataagcctaggatacattttgtgccaggaagcaagaagatgttcaaatgatttccaagtaatgtttggaaatgatatttgaaaatgatttccaaatgatatttccaaatgatttccaaatgatatatggaaacacttaaagactccactaaagaactattagatctgataaacaaattcagtaatgttgctggatacaaaatcaacatacaaaaaccagtagcatttctgcatgccaacagtgaacaatctggcaaaaataaaaaatgtaatcccatttacaataaccccaaataaaactaaatacctgggaattaacttaagagaaagatgtctacaattaatattgtaaaacactgatgaaggaaattgaagaagacacaaaaaagaaggatattccatgtttatatattgtaagcattaatattgttaaaaatgtccatactacccaaagcaatgcacagattcaatgcagtctctcaaaataccaatggcattcttcaaagaaatagaaaaaaaaaaaccctaaaatttgtatggaaccacaaaagacccagaatagcgaaagctaccttcagcaaaaagaacaaaactggaggaatcatattacctgacttcaaattatactacagaggtataataaccaaaacagtatggtacttgtataaaaacagacacagaccaatgaaatagaatagagaacccagaaacaattccacacacctacggtgaactcattttcaacaatgttgtcaagaacatacactgggggaaaagacagtctcttctggtgctgggaaagctggattttaacatgcagaataatgaaactagaaccctgtatctcaccagacacaaaaatcaaatcaaggtggacgaaagactgaaacctggctgagtgccgtggctcatgcctgtaatcccagcattttgagaggccgaggcgggtgtatcacttgaggtcaggagttcaagaccagcctggccaacatggtgaaaccacatgtctaccaaaaaatacaagagttagctggacatgctggtgcgtgcctgtagtcccagctacacagaaggctgaggtggcagaatcacttggacccaggaggcggaggtggcagtgagctgagatcatgacaatgcaccccagcctgggcaagagagtgagactctgtcagaaaaacaaaaaacaaaaaaacaaaaaacaaaactgaaatctgagacctcaaacgatgaaactgctacaagaaaacattgtggaaactcttcaggatattggtctgggcaaaactttctgaagaactaccccacaagcacaggcaaccaaagcaaaaatggacaaatggatcagatcaagttaaaaagcttctgtaccacaaagaaagcaatcaacaaagtgaagacacaaaccacagaatgggagaaaatattttcaaagtcacactctgacaacagattaatagccagaatacatgaagcgctcaaacaactctgtaaggaaaaatctaataatccaatcaaaaaatgggcaaaatttgaatagacatttttcaaaagaagacatacaaatgccacataggcatatgataaggtgctcaacatcactggtcattagagaaatgcaaatcaaaaccacaatgagatatcatcttaccccagctaaaatggtttttatccaaaagacaggcaacaacaaatgccagcgagaatgtggagaaaagggaacccttgtacactgttggtgtaaattagtgcaaccactatagagaacaatttggaggttcctcaaaacattaaaattaacattaaatagagctaccacaatatccagaaatccccatgctgggtatatacctggaagaaaggaaatcatatattgaagagataacatcactccaatattcacaatagccactattcacaaatgccaagatttggaagcaacctaagtgtccatcaacagatgaatggataaagaaagtactccaattatacacaatggagcacaattcagccatgaaaaaagcatgagatcctgttatctgtaataatatggatggaactggaggtcatcatgttaagtgaaataagccaggcacagaaacacagatattgcaagttctcacatacttgtgggatctacaaatcaaaacaactgagctaatgtctgggccttagtcagtgttgtacccaagtactgggagcacagcttttaaaatacatcatgaatgctttaatacaggaatgaatagatgagaggcacaaactggttgggtgttcttctgatacacagtatcttccttgacagattcagtacaactctcaacaggtaagtctcttcatgttatgttaccttatgaggaattaagtggcagaacatgatttctattattttcctttgcagaacaagaccaactttattagttgggacacagtgtggctgcatttgagtcccaagcaaccattagtctattgctatcaccacagagtcagaggggatgagacgcccagcaatctcacccaagacaactccaccaacattcctggttacccaccatgtgtacagtaccctgctaggaaccagggtcatgaaagtaaataataccagactgtgcccttgaggagctcacctctgctaagggaaacaggcatagaaacttacaatggtggtagagagaaaagaggacaataggactgtgtgagggggataggaggcacccagaggaggaaatggttacatttgtgtgaggaggttggtaaggaaaaattttagcagaaggggtctgtctggctgggcttggaaggatacgtaggagtcatctagagggcacaggtacactccaggcagagggaatttcgtgggtaaagatgtgtaggtgtggcttgtgaggatggatttcaattattctagaatgaaggcagccatggaggggcaggtgagaggagggttaatagatttcatgccaatggctccacttgagtttctgataagaacccagaacccttggactccccgataacactgattaagcttttcatgattcctcatagaacatgaactcaaaagaggtcagcaaaggggtgtgtgcgattctttgctattggctgcagctatagccctgcctccttctccagcacataaatctttcagcagcttggctgaagactgctgtgcagggcagggaagctccaggcaaacagcccagcaaacagcagcactcagctaaaaggaagactcacagaacacagttgaagaaggaaagtggcgatggacctcatcccaaatttggcggtggaaacctggcttctcctggctgtcagcctggtgctcctctatctgtgagtaactgtccaaactcctctctttgtttccttggacttggggtgctaatcgggccccttttcccttatctgttttgaagatcaaaagagatgttcaaggagaagtagctgaagtgttggacgctacaaacgcatagaagttattattatcttatgcagatctatgaatgaataaataagcatttctcccatccaccttctaattttggtgactaggagggtttagggacagcatttggtagtgggaatgatttgattagcttagatctgacgaagactaatcaatgaaaacatggcagcggcagattacaaactgctgatcatgatggacagtgtgatcctcatccccttcccaggctctggggattctgggtacaggaaggagtggcttgcatttttgtctcattaattcgctttctgggttctgtgtctgctggaagggatgtgtagctgtattgcccctgtagacctggttcctgctcccccgccttccaacccaggatatcatttacataacgcaccaggggacaccaagacttcatgggaagctgtcccctggctcttccctctttcctgtgccatgcccctgaaaatcccctccctcctatgagtcactcctccaccctgtcatacacaggatggtttatcttgcaatgattaacctctagagcaaaggagacctggaggaagtttcgaggatttattctttgctttaatctttttcctcccgtctctgggaggctaggattaatatagagctttgtttctcacctaatgggaatctactagcagcctgaaaaggcaggagccatgaaagccaatttggattttacatatttttcccctttatgttacagtacaggagggcaaaccctctcactggtgggattcctggcatcctagagcaggtggagagaagagttactttccactgtgggtagtggaggctccacctgtcccattaacttctacctcaatttgacttttattaagagcagggaaccacaatgacatgaaaatagacactataaacctcattttaattctttcacagaaagcttaggaattcagtgagttgtggcaacatggtttccattgtctaacatttttaaatgaattgatatggtttaaattcattcatttttaaaccagaattttttggagatagactatttccagcatgttccttctggatggtaaaacagggctgttagttcagtatttgtgacaataagtgtgtgtaaaataatgtcacctttcctgaatgtcaggaatatgagtctaatgcacaaatgtatacctctaagacaagactgcacgtcttttcaaatatacctgtccggccatttattttaataactccttttcgaatatacctgcttagcagattgtcttaaactctcaggacaggggagtaagcaagactgtgagccagtgacgatagcaaaggcttccaggtaggatccatatgaagtgagaaaatattcctcagctctcagggtagaactccaaagagatattcatgggtcctggccccaccgtggaggtcactcaaagggcaaacaggttggcatctcatctgcttcaagcctggacacaggggcaccatctgtgtcactctgtgtgtggtctgccatgttgtgggccggtcactacagactcgggcagccaggcagacaatgccttagccttagacaatgctggtgcagcccaggagtcagaaaatgcagtgtagaccaggccctccttaggccaacacaattacatgcaatagatgactggcttttctgttagtctcttcactggacccaaaggctgcattactctaccagaggggagctggaaagaaactaaagagttcgcccagcacagcatctgccttgacatggtaccatgtgaatctagacactcaccaagatctttccttgggggccaatgctgctgacacattaactcaatagcttgtcctcacctgagaggtcaggtaatgtgtttaaagttcaggagcagagattagtgtcattgatttgacatggctgtgacaacaaaggagggaactgaagtgggaatacccaaggccaccctggctttggcaggtggtgcacgcacttccactaactgttctggggcagggaaccaaatgtatgactgggcctgctcatgctgcccctgctgagtcctccaaaccctgcccttcatgtaatttctcagttttattttatcacattttataagtcactggatgtttacaaaatgtttggaacctatactgccttgaaggctaacctctaaagaggagtaaacaaggtcttaatacaactctccgggacgttttatcattacttatcttatatgccatactgcaccatttgctatcaacaggaaagtacctggactttggaaggtccctctgtgtcttttagctgaaagtacatatgaggcatgtggattcttttatgcacatcatctttttcagccacatttttgtagtttgcctctctggagccaactgtgtggggctagcagcttcacagctgaatcagtgtctggcaacctcttccttcagcctctcttcttcctccagttttccatccctcagtcacaccggagggggaaggtctgcaaggatccagaaccatcagttggaggagtttgcacatgactcatgaaagatgagttccaggcaggcctgccatagtgaacaccaggcttaatgggtttttcctcagagatacttcacgtacagaggcagtgaactgactgctttctggttgaccaccttgaaaaagatgagtgtgcctggcactgtgcttctcaggtgagtatgacctgagaagtattagttgctggttcttctgcacacaatcattcaaggacatatggatcaaccatcctcctcaacagctcaaatcaaccagatcatctgaccacagagactgaggtgtacctgaaagctgcccacatttctataaggccaatagaagccatgaacacagttgtcaatctgtagaaataaggactccatgactcctccaaggcctctctgtgaatgaacgtttaagaagggctagatcctaaaacagggtcagagcttagagggaagaaaaagcataaacatttctgagcaaattgtaagggcagtgtcaccataggctcccagtgaccctctgtgattgagtgcatacagtgatgcaaaatctcatcatcagtgcaaaagacaaaaaaaatcttactctttctacctaggatgagagtccccaaatcagcgaagagtccacttactaaacagacataaggaaatgaagtgtcctggaagaattcctgcctgaacctctcaggagcatttgaggacatttatcaagtattcactccaggattgggactatgaagacttcagctgctttcagctaatcattgagacttttcaggggtctcagaatagtcaggaaaggacctgatgagtgaatgcaattactgatgttggagttgctgttattatttatcgtgtacatattacctccctctcttgaccattccagttcctgagtaactcaccagccctctgatctataaagtcacaatccctgtgacctgatttctgtttcactttgtagatatgggacccgtacacatggactttttaagagactgggaattccagggcccacacctctgcctttgttgggaaatgttttgtcctatcgtcaggtgagttgcttgagcttcctcttttgcttcttatggttgcaaacatcagcttagttccatcagtaaaaatgcccctccttgggagggagttctgaggtttcacattttcagaaatggtgggactgggtgcagtggatcatgcctgtaatctcagcctctgtgaggccaagactggcaaattgcttgagcccaggagtttgagaacagcctgggcaacacagtgagacacctgtctctagaaagaaaaaattacctgtgcatgatatggtagcccatgcctgtagtcccagctactctgaatgttaaggtgggaggattgtatgaacccaggaagtcaaggctgtattgagctgtgatcgcaccactgcactccagcttggtcaacagaacaagacagaaaggaagaaagaaagagagagagagaaagaaagagagaggaaggagaggggaggggaggggaggggaggggggaggagaggagaggagagaaaaggagaggagagaggagaggagaggaaaaggtgtgtaggctccacccaaagcatggccaggtttacccctggagggaaagtcacaagctcatgtccagaaggccagtagcagcaagctgctctccagcccagatttcctatcctgtgtacctggagcttgtttctcagattctaactctcacaactgaagcctctgttgtctgattactatctgagaattctacacaattttaccctcgataaaagcagtaatttcttcttcatctttcccagatcaactcttgtagtagatcaacatttctgggaccttcttttgcatggttaaaacatcacagctgaatcttagcaacaggaaggtttgtttttatgtttcagaagtgaaagctcagagcacgcattgtaatttgctgggtgtgatgtgtagaggtggcatttctccatcttttctgtgttaagctagaaaactggaaaggaagtctactttctcattcactcactcactttctcactcaacaacatgccttagacttatctaaatctgcaagactaaaagaggttcctggtttctttaactttctaattctgctagagttctagagagagcacatgagataaatgaaaaggatactgatggaggagattaaaaaattgtgcattccctgcagacactcacttttcctcacctcagtttcacccctgcccttgcaggtgatcattcacggggttaggagactttagagagaataaaagaaaaagcaaaaatacatcagaaagacaaggaattacttactggtcatagacaagggtgagtccttcagtacttagagaaaattcaagagtgactttaaattccccacttcaaatatattctctgttttcttgtctttcccttaagacatctctgaatagcttccttcaactgccagtgaaagatagcaggcctgatttcattggacgcaactgttttcagccccaattagaggtagggtttattctatttaaaataataatcaacttgtattttgtttcctctcccagggtctctggaaatttgacacagagtgctataaaaagtatggaaaaatgtgggggtgagtattctgaaaacctccattggatagacctgctactgtgaggaggttaccccactgcaggatagtctctgcccaggtcttcatgggatgaagctcttgtcaacctaaatacaaacagagagaggttctctgaaagaagaggataattacttgggagtagaatattgcaatgggaatctgcttgccgttataaactatgtgcaaattcagggaggtaaacaagacaaagatgctccatagaaaatatgagaagaatctcataactgttttgagataattattgttagctacaaagatcaataacaagggtgatgccacaccaaggttggacaggcagttgctggacaggtgtccttgcagaaatatttttgtgtaaagttgaaatagcctttgtgcaaagttgtggtttttgtagacacttttgtaatagttttgtttccaggaacacaagcataagaatcctctcttcatagccttcttgggatttatttgtcagggttaaaaaacaattagtgacatcactttggttctgataaagttcacactcgctattgtaaaacttttcgaggcttgtcctaccaaggatcccatgtgtcaccaggtatcgaggtcttcagtctgaactaggctaggagcattgtggttaccacttttctgcaggttttggtggcccagggactcccagcatcgccttctgtccagtgtctgcctattcccctcttctttttttcttccttaggtgcccttttatcacatgcattgtctcagacccttctaatatgtgctcataaatgcatggcatcatctccttcccacattgattcactttcaattaaaagccaaaactccttcatttagactgaatttaacatgtgcttttgaaagaagggttgagagataatagagaaacagattgggaaaccacttatgctccacttttttaaactttctctgcaagtatggaattttttgttctgctttgttgtttaaatttaagccaaaacttcttaatagaaggatatacaaatatttattggtttataccattgcacttactttgaagaagagatgctgaatattattaaaccattgtgttccctggtgggctgatggactgtgattttataaggtggtctcagccaattgcagcagctgttccctgtcagaggggctagaggtttggtgagagcagtggatgaggtgcagtggtgtgtttgttcactagaagcaagtgggagaaagctttgcctctttgtacttcttcatcttctcccctcaagtcctcagaatccacagcgctgactgtggagtgctgtggagctggcatggcccatacaggcaacatgacttagtagacagatgacacagctctagatgtccatgggccccacaccaactgcccttgcagcatttagtccttgtgagcacttgatgatttacctgccttcaatttttcactgacctaatattctttttgataatgaagtattttaaacatataaaacattatggagagtggcataggagatacccacgtatgtaccacccagcttaacgaatgctctactgtcatttctaaccataatctctttaaagagctcttttgtctttcagtatctcttccctgtttggaccacattacccttcatcatatgaagccttgggtggctcctgtgtgagactcttgctgtgtgtcacaccctaatgaactagaacctaaggttgctgtgtgtcgtacaactaggggtatggattacataacataatgatcaaagtctggcttcctgggtgtggctccagctgcagaatcgggctagtgaagtttaatcagctccgttgtccccacacagaacgtatgaaggtcaactccctgtgctggccatcacagatcccgacgtgatcagaacagtgctagtgaaagaatgttattctgtcttcacaaatcgaagggtaagcatccattttttgaaatttaaataatgattgatccactgattaaatttttattttgaaaaaaacatatattcacagaaggttacctaaaaaatgtacaggaaggttccatgtactcttcatcctgtcccgcccagtggtaacatcttgcaatcttgtatattgcaatatatatctagtatattcatattatcaggttggcacaaaagttaaaatggcaaactacaggctgggcataatggctcatgcctgtaatcccagcactttgggaggccgaggcaggtggatcacgaggtcaggagttcgagatcagcctgaccaacatggtgaaaccccatctctactaaaaatacaaaaattagctgcgtgtggtggcatgcgcctgtagtcccagctactcagtagtctgagacaggagaatcgcttgaacctgggaggcggaggttgcagtgagccgagatcacgccattatactccagtctgggcaacccaatgagactccatctcaaacaacaacaacaacaacaacaacaaaaaccggcaaactgcaataacttttgcaccaacctaatactatagtacaggaaattgactttgatatagtttacagagcttttcagatttcaccagttttacatgcccttgtttgtgtgtgtttatgtgtgtgggtagttctaagcaatttttcacattcgtagatttgtgcaacgaccagcaccatcaagatgcagacccattccgtcaccatgtggctccctcctgctgtcctacagtcacaacatggagtttgtctttttctctgacaggttctatatcagagcaaacttttatttatttgaggaggccaatgtattaatatttccttttatggattgttcttttggtgttaagtctgaaaatcctttgcttagccctccttcctacattgctttttctaagagttatatagtttaacactttacaaaatgtaactctattacccattttgtgttaatatttgcataagttatgagatttagatcaaggttcattttctgtggactatggctgtccaaatgttccaacaccattttggaaaggtaggcatattgtcaaaactcagctgagtatattttgtgaatctatttcttattgtttactcctccactaataccacactgtggtgactctagtagctgtacagtaactcttaacatcatatagggcaattctttccactttattgatttatattttcagaatggctttagcttttcttgtcccttgcctttccataaaaattcagaataagcttgtaagtgtctacaaacaaacctgccataattttgataagaattaaagcagaggtgtccaatcttttggcttccctgggccacagtggaagaagaagtgtcgtgggccacacataaaatacacacacacacacacacacacacacacacacacacacacacacacaaatggtctgtgtatagttttcattatatatctaccaccacagataagcaaaaatgtccttgcataataatcctaattatgcactgccccattcagagggtctttcaaaatcattgaacaggttccaagtttgcaatcactgatacagaaaatgtacatatctagctaaacttcactacttttttgatattttttattataaaagaaaagagaacaacataaaactagtggggtacttgacattgtttttgagaaactaatccatcagtatctggcttgatggaagtagttgcaattctcagtgagttctcaaggtgctcatcagatattttggttctaattttactcttcgtgttcttcatccttgaaaatagtagctcacaaatgtaagtgctgccaaaaagcaatgacatgaacaaggtgtgattgtgaagcaagggatatttgtcattgggaagacaggtcttacaaaagtccagtaaagaggcaaaatcaaatttttctataagttgaacatcagattgcagctctaggcattccatttcaaaattgccaggtaacatatatatgtcgactgaaaatggagttgcaaatataccaaaatattgatgattttttcagaaatcttgaaatacctgttttcaaattcctgtatcaaattgaaaagcaaggctgcgtatttttggctgttcacaggaccatgtttagccaacatgtcgaaatgcataaaattgtttgccttaatttgagcttgccataatttcagtttcatatggaatgctgttatggtttgaaacattgtattgttaagttggttttcaacttgaagacacaggtttaactcacttaaatgggccgtcaaacccactaaaaatgctaaatctgtaagccagttttcattgtcaagttctggcaccaattttgtttgataccataaacagcttgatttcacatcacaaagcataaaatctttacattttgccttgacttaaccatcttacttctaaaaagtgaatgacttgctagagtcagcatccatacttttaaggaattcctgaaactagcgatgattcaattcctgggcccttgtgaaatttacagccttgatgacaatttgcatgacgttatctacttttaaagcttgtgcacatggattttcttgatgtattatgcaataatacttcatcaaatgtgagttttgtgtggcaactgcatcatctattaattgtacaagtccctctcttttacctaccatcgccagggcagcatctgtagctatatcacatatgtttacaaaggacaaagaaaattgctttaacatatttttcactgcttcatataaatctcttgatttagttgtgtcttttaatagcatggtgacatttcgatttcttcagtgacattatattcatcatcaatacctctaataaaaatagcaagttgtgccgtatctgtagtgtcagtgccttcatccatcaccaaagcataaaattttaaattagcagttttactctccaaacgtctttcaatagatttcccaatttctccaattctcctggctatagtctggtgagacaaactgattttagaaatatcagtttctcaaggcaaataatatctaccacatcttccagacattgcttaataaactcaccatcagtaaatggttttgatttttttgctattaaatttgctaccacataactaggttttaccttacgattgagtccgagttgtaactttttaaaaaatcttttttgttgaaaagacagactttttttcagttctgctattttgtccttacaacacatacacaccaaatttgtcagcacgtttttgcatataatgcctcttcaaattgtagtctttgaaaactggcacaaattccgtggaaattaagcagagtgcttcgctatttgcctcaacaagaaaaagtcatttgtccacttttcattgaacaatcttccttcatccataatttttgttttttagggttttctttttaagacattgtggaagccattctggaattaaaagcattataatagataagcaactatatttacttttattatggaaattaacagataggaaaatagaacagaaagcaaggtttaataatcaaataagaatacttacatgtcttctaaataatattaaacacctatcatctacaaaggtaggttgaaatattattgataattgctgggttttacttgccaaattgccacaaacacacctaatacctgacagtgtcaattcaactgtccgtgattagaagataacacactggaagtcgcacaccaccataaaactgaagccacacatgcgtacaaatggcgacagtgtctggtgtacagcagcgctctgccttgtccagaatacacacttgaattctttgtcacaattcacttcacgtggcactgcaatagcgtcctctcgctctttgttagttaattttaatggcttttaatttcttcttgctgaactgtttgcaattataatgcaaattatggatactagtccattatttgtggatgtgacatactctgattacccctttccattccattgttgtctacgaagttcacacttgagaatcacatagtcaaattacaaaattacaaaaaaaattgcaaaaaaactcaaaatgttttaagaaagtttccacatttgtattgggatacattcaaagccatcctggactgcatgaggcctgcaggccacaagttggacaagcttgaattaaaccaatagaacaatttgggtataatctatatctttactatgttcagcctttcatcccgtgaatatagtatgcctctccatttctttagcttttattactttcctcaacattttatagttttcagcatagaggtcctgtacatcttttgttagatttacaccagaaatatttcatttttgttggagtaactgtaaatgatactgtttttcttgtattttcagatattgattattgttacatagaaatgtgaataattttgtttgttgatcttgtatcctatagccttgcagaacttacctattcgttctagaaatttttttgtatattccttgacattttatacattgacaattatgtcacctgaaaatagagacaattctattatttcctttccaatctgtatgccttttatttctttttcttgtctagtgtattaagacatcaggtatgctctttagtaagaatgttgagagtgggcattttttagttcttcttgatcttggaaaaaccattcagtccttcatcattaaatgtgatttaactgaatgatttttttacagattgtctttatcaaatgaaggaactgtctctctcttcctagtttattgagattttatcatgacagctggaagtacacattttaaaacaaaacatagttgtggaagataagagaaagttccaagcatgctggcttgatagtccagccccaagttgggaaaagtaattatccctttctttttccttctatttatggaataaaaaattaagagaaaagaattttcaaggaaattgcattattccttcaaaacaggtttctagtctttaagtattacctacttttcaaaaaaaaatcaccacatcatggcatccctttttcaagttgcccatgctgtaggtgtattaaagacagagctggtctgaggcaacatacagtctgcccatctgtcaccaatccttttctactctgcacactcctggggaagggctaggtcttgttcctgtctattccactggaagaacagttccctaccacgtggagcatttgcaattaaaaggagactgagatatagaggcaggagaccacaccagatggctgggtctccccactcccacccccgccccacatacactcagaagaggctaggcatctaggatctccattgagcatcttgaatatggcttgccataatatcatatacagtcaataaatatttgttaaataaggatgcctcttcaatatattttgtgcaaccatgaagatcaccacaactaatgtgagaaaaaatgtttctgttgaactctagtctttaggcccagtgggatttatgaaaagtgccatctctttagctgaggatgaagaatggaagagaatacggtcattgctgtctccaaccttcaccagcggaaaactcaaggaggtatgaaaataagatgagtcttaattagaaatgtaaagaatgaatctggggacaggtagaaagtaagatcacagtccgtttccaaggggtagtccactgagttcgagcttcctaaaaatggtcttttatctttatgtacagaaaagacatcacaaaattcattacaaaatgtcacttactgctccatgctggagaaagccatatccttctgggacttgagtctgcacatttaactacaggtactgatctgttttgtgcttagatgttccccatcattgcccagtatggagatgtattggtgagaaacttgaggcgggaagcagagaaaggcaagcctgtcaccttgaaagagtaagtaggagcacagccatggggttctgagctgtcatgagcccttccagctgcctgccatggagtcgacagtcgcactgttgggttactccagtgaccagacaaaagcagggcagcgctgcaactccaaagagccacctaagagggagtggctcccatgaggcggcaagtcagcaagggaaaagggccttctctcctgtgcacaggagccaggatttacttatctgttaacttgtcaccataaatattctgggagattaaatacatactttagaaattaaaaaaacatgattgtatcaaagttttgagtgtagtggatatggaactgtgggtaagcaagcatttggtacttgttgccttgcattgggtaagatgggaaagttacaatggggaacttggaacaatttcaatcccttcatggtttttctgagaatatcagcaaactatgaactattaaaccttcccactacttccttttcctccaatctcaaaaaagaaagggtgctagaaatgctatgtgtagagcaagcctattatttgctgtctacaatggtatgtgcttcaattatgcaggaacgacaggtgtaatctgagcctgtcctgttcagacttgggacatgtggtcactcagttttgggttctccaaatcaatgttggagagatctattttttttaaccagaacattcttgattgtcacatcttacaaaaatgactctgctctcagcgcaacttcaggtcagaggagctggggatagtggggttttccagagcattagcagggagtgtagagaataaaggatgatatttctaggaactcagaacagggtgttactgttttgtaaagtgttgaagaggaattggctctgggcatagagtctgtagtcagacaacgccacctttcttgaatccactaggaagagttaattattctactcttgttctgctgaagcacagagcttacatatcttatatcatccacactcaacacatgctactgtagttgtctgataatgggtctctgtcttcctatgactgggctccttgacctcagaggtgagtctaactcagcttggtgtctccatcacccccagcatagggccagctccatcactggcaccagataaccaccttctgagggagtagatggaagatgattcagcagatagttctgaaagtctgtggctctttatgtgtcttgactggatatgtgggtttcttgctgcatgtatagtggaaggacggtaagaggtgctgattttaattttccatatctttctccactcagcatctttggggcctacagcatggatgtgattactggcacatcatttggagtgaacatcgactctctcaacaatccacaagacccctttgtggagagcactaagaagttcctaaaatttggtttcttagatccattatttctctcaataagtatgtgggctattatttctttctctctttttaaaaataactgctttcttgacatataattcacatatcgtataattcatccacttaaaaggtacaattccattgtttttaagataatcaaaaatatgtatgaccattactattgtaaactaaaatgtttttgtcaatctagagccctcacacactttagctgtcaacaccccaccacaaaccccactgccctaagcatccaataatcaactttctgcctctatagatttgcctattctggacacttcatagaaataatatcattgatttttctctgttgttttttattctctatttcatgagtttattttagtctgttattttctttcttttgctggctttaggtttcatttgctcttcttcttttagtgttttgtggtgtaaataattataatcaatttgagatattttcttcttttaaatttagatattacagctataaatttccctctgagcactggtttggctacatcctgtgttttggtacatcatgccttctttttgttcatctcaaaacaatttcttgttgcccttttgatttctgctttgactcactggtcacttaaaactgtattgtttaacttccacaaatgtatgagtttcccaaatttctttcccttattgatttctagttttattccatggaagttgatgtacatatgctgtgttaattctatcttgactatcatttcctgaacagcatgattaagttaagcagcagattatggtctacattaatccaaaaactctagtccaatagataaaggctaagaggtcagggaatttaattctattactttggtcactccaaagactcagaaggtgccattgatctcactgctgtagtggtgtttcctatgtatagacctgcccttgctcagtcgccggcctgaaagaagggcaaacatgataaaaggaatgggttccagttgagaatcatgatgttcttattcttattactggtagagaaaattataattgctccaggtaaagtttgcattttcaatgatttccttttgtttgttttgtttttcccacagtactctttccattccttaccccagtttttgaagcattaaatgtctctctgtttccaaaagataccataaattttttaagtaaatctgtaaacagaatgaagaaaagtcgcctcaacgacaaacaaaaggtaaaatctgatggtggttaaatgacgatgtttaggttttgataaatttagattttatacacatgatagagcatgtatctgtatttttaaaaataaagacagagaacttatgtttagaacaagagaagccatttggtagaaataaagaaggagattggggaaggagatgagaatgagtcagagagatagcatttaaaacttgaaatcaggcacaacaattagtatgtcatgatataaacagtattgagataaaattttaccacttctcttccctttaataaattgtcaaaggataaagtttcctgtttgaaaatatattttactggtattgtgctttcctcatatcacagattggtaaagaatcattttaagtccaagactcttattttacatattctgcaattaaaggtcctatgaggctacctgccgactgctgacatgtagtgtgtggtaaatgtgagtgtttcacagcctggagtgaacaggggtcttctctgagaattgaggttgcaaggctggctaactcagctttgccttcacgagccctagaggccagccgaaggatgtctgcaggtcagggagacaggaccaggtaacccagctgtcactgaagattatatagagtttgagaatgttggaatatttgaaaatgctcccccaaaaaagctgctgatgagttctggaaatgtcaggagattaatctatacggacactgctgaagaaaaaggtagaagaataaaagatccagtacttcttcctgggtaagcagttatgaccagagatggaaccggcaactctttggccagaaagctgtatccaaaagacagagaagatgagaaacagggagggcaaaggcgaaaaagcaattggacatgatagctagatttgtttcaggaaaacatcctgctttccaaggatttagatgaatgtttttgttcactggtgactcaggtaacacgtcttcaagaagccatagggaggttgagggagggaagtcaagaagggaggttgaggactgcacttttgatttacttctgacttcacgagtcactttctgccaaagaaatctctccttttgcttctagcaccgactagatttccttcagctgatgattgactcccagaattcgaaagaaactgagtcccacaaaggtaaccaaggagtgcttctgagggctactggcggggacactaagagggagggccttgttctgaaaatgtgcaggaagtattccaggaagatgagaatttttgccacatagcagaacaacacacatttagatgttataaatggtagctggaggcactttccagaagcccacaggtatagccatgttccaggctgaaagggcaaccctaagcaaacctagaatgcttggaggacagtcagtggtttgtggatcacctacatgagatcaaatgccagttctcagcctcctccagatccaccaagtgagaacctctacttggaaatttatatcaaacataccgatcaggaagcacactatcccagtaagggtgattttaactggcagtacttgaaagtgtgttcgcaaggttaatctactgcaaagttttatttttccctttgaaatgcataagtaactaatgggggacacctctgataccatgtaaatctacttcaatcttcagtcttgtatctactagttttatgacccatggatggttttaaccaaaaccattattactaagacagtggcaaaatgataaccatggtcaatttcaagctaccaagatttggcaaccatctcacaaaatttttgaatatttaacaattggttctagagagcaggactcagcagactccagtataccactttaaacatgtccatgtctacatctacttctgtctgtctatctatctgtcaatcatctatctgcctataatttatcaattaatcatctatctatctcaacaaaacttgctgtgataaagaaaatagtctatcatttcactgtttcatatagaaatcactagacacatatggctattgagtactggacatgtggccaatgccactgaagaacaatttttaagagtatttatttttaattgaataaaatttgaatttaaatagccacatgtggatagtggctaccagattggacagcagagctcccaactttaaaattacagttcaatttcaactcagtataatggggttcaatgtaactgagtaaaataattggatggttgaatttacccacagcagcatacagaaatattcactgataaatcagaactctgtagacctttctcacactcattttatattgtgtttggttgtgagttacatgattgctgcaggcaccatatttatttctgtgctccaggtctctaaaggtcctaatccagtcctgaccaaacagactagtgatggaccatcgtgagcttctctcaggagaaatatcaagagggaggccaacctgtaatcataagaacttctgctattttaatgccattcatcagactacagtcaatcaccatgcttctggctttttgtctatctctgctgtcttgtacatcctgagatagtccattctgagaactgtaccctagatcttgtattgcctgatgcctgtcaaagatgtaatccatgctgcttaagtgaggttgtgcacacaaatcaccatatctcctgcaagtttggattttgattcagtagttcgatggtggggtttgagattctgcatttctaataagctcccagatgtggctggtgctgctggtccatgaaacacactttgagtagcaagaggtgatctgtagctcagtattggtcctttaagttccctcaaacatatatagagaaaaggtcctaaatattgcaaattctctcaaagtttgtcaagctatattggaattctctcaaagtctgtcaagctctattgtagaaaatcaaatttttattgggaaaaagcctaccccatatttacttacagataaagtacttttaggatcattcaaggcacacacccataacactgagtatgtaagacagaaatgctctctctggaaattacagcagtgctggtgctgggatgccatgatgaggagtgtgtggcccacaatcatgtagaccttgggaaaacctggattaaaatgattttgcgtcatcctggccctgtataagatacatatcagaatgaaaaccactcccagtgtgactttgaattgcttttccattttttcttcttgggattagagagcttcacttagatttcatctaagctgtgatgttgtacgttgacctgatttacctaaaatgtctttcctctcctttcagctctgtctgatctggagctcgcagcccagtcaataatcttcatttttgctggctatgaaaccaccagcagtgttctttccttcactttatatgaactggccactcaccctgatgtccagcagaaactgcaaaaggagattgatgcagttttgcccaataaggtgaggggatgacccctggagatgaagggaagaggtgaagccttagcaaaaatgcctcctcaccactccccaggagaatttttataaaaagcataatcactgattccttcactgacataatgtaggaagcctctgaggagaaaaacaaagggagaaacatagagaacggttgctactggcagaagcataagatctttgtacaatattgctggccctggttcacctgtttactgttatcacaataatgctaagtaaaaaaaaaaaaaaaaaaaaaaaaaaaaaaaggagtgtggcgagaagatggccaaacaggaacagctccagtctacagctcccagcgtgagcaacacagaagacgaatgatttctgcatttccaactgaggtaccgggtgcatctcaatggggattgttggagagtgggtgcaggacagtgggtgcagtgcacccagcctgagccaaagcagggcgaggcatcacctcacctgggaagtgcaaggggtcagggaattccctttcctaggggtgacggacagcacctggaaaatcaggtcactcccaccctaatactgcgcttttctgatggtcttagcaaacggcacaccaggagattatatcccgcgcatggctcggagggtcctacgcccatggagcctcgctcattgctagcacagcagtctgagatcgaactgcaaggcagcagcaaggctgggggaggggcgcccgccattgctaaggcttgagtaggtaaacaaagctgccaggaagctcaaactgggtgaagcccaccgcagctcaaggaggtctgcctgcctctgtagactccacctctaggggcagagcatagccaaccaaaaggcagcagaaacctctgcagacttaaatgtccctgtctgacagctttgaagagagtagtggttctcccagcacacagctggagatctgagaacagacagactgcctcctcaagtgggtccctgacccccgagcagcctaactgggaggcaccccccagtaggggcagactgacacctcacacggccgggtactcctctgagacaaaacttccagaggaatgatcaggcagcagcatttgcgggtcaccaataccgctgttctgcagcctccactcctgatacccaggcaaacagggtctggagtggacctccggcaaactccaacagacctgcagctgaggatcctgactgtcagaaggaaaactaacaaacagaaaggacatccacaccaaaacccatctgtacatcaccatcatcaaagatcaaaggtagataaaaacacaaagatgggggaaaaacagcagaaaaactgaaaaatctaaaaatcagagcacctctcctcctccaaaggaacgcagctccgcaccagcaacggaaagctggatggagaatgactttgacgagttgagagaagaaggcttcagacgatcaaactactccgagctaaaggaggaagttcgaacccatggcaaagaagttaaaaaccttgaaaaaagattagacaaatggctaactagaataatcaatgcagagaagtccttaaaggacctgatggagctgaagaccatggcacgagaactacgtgatgaatgcacaagcctcagtagccaattcaatcaactggaagaaagggtatcagtgatggaagatcaaatgaatgaaatgaagaaagaagagaagtttagaagaaaaagaataaaaagaaaggaacaaagcctccaagaaatatgggactatgtgaaaagaccaaatctacgtctgattggtgtacctgaaagtgacggggagaatagaacgaagttggaaaacactctgcaggatattatccaggagaacttccccaatctagcaaggcaggccaacattcaaattcaggaaatacagagaacgccacaaagatactcctcgagaagagcaactccaagacacataattgtcagattcaccaaagttgaaatgaaggaaaaaatgttaagggcagccagagagaaaggtcgggttacccacaaacacaaacccatcagactaacagtggatctctcggcagaaactctacaagccagtagagagtgggggccaatattcaacattcttaaagaaaagaattttcaacccagaatttcatttccagccaaactaagcttcataagtgaaggagaaataaaatactttacagacaagcaaatgctgagagattttgtcaccaccaggcctgccctaaaagagctcttgaaggaagcactaaacatggaaaggaacaactggtaccagccactgcaaaaacatgccaaattgtaaagaccatcgaggctaaggagaaactgcatcaactaacgagcaaaataatcagctaacatcataatgacaggatcaaattcacatataaaaatattaaccttaaatgtaaacgggctaaatgctccaattaaaagacacagactggcaaactggatagagtcaagacccatcggtgtgctgtattcaggaaacccatctcacgtgcaaagtaacacataggctcaaaataaagggatggaggaagatctaccaagcaaatggacaacaaaaaaaggcaggggttgcaatcctactctctgataaaacaggctttaaaccaacaaagatcaaaagagacaaagaaggccattacataatggtaaagggatcaattcaacaagaagagctaactatcctaaatatatatgcacccaatacaggagcacccagattcatgaagcaagtctttagagacttacaaagagagttagactcccacacaataataatggaagactttaacaccacactgtcaacactagacagatcaacaggacagaaagttaagaaggatatccaggaattgaactcagctctgcacaaagtggacataatagacatctacagaactctccaccccaaatcaacagaatatacattcttttcagcaccacaccacacctattccaaaattaaccacatagttggaagtaaagcactcctcagcaaatgtaaaagaacagacattataacaaactgtctctcagaccacagtgcaatcaaactagaactcaggattcagaaactcactcaaaaccgctcaactacatggaaactgaacaacctgctcctgaatgactactgggtacataacgaaatgaaggcagaaataaagatgttctttgaaaccaacaagaacaaagacacaacataccagaatctctgggccacattcaaagcaatgtgtagagggaaatttatagcactaaatgcctacaagagaaagcaggaaagatctaacattgacaccctaacatcacaatgaaaagaactagagaagcaggagcaaacacattcaaaagatagcagaaggcaagaaataactaagatcagagcagaactgaaggaaacagagacacaaaaaaacccttcaaaaaaatcaatgaatccaggagctggttttttgaaaagatcaacaaaattgatagaatgctagcaagactaataaagaagaaaagagagaagaatcaaatagatgcaataaaaatgataaaggggatatcaccacccatcccacagaaatacaaactaccatcagagaatactataaacacctctatgcaaataaactagaaaatctagaagaaatggataaattcctcgacacatacactctcccaagactaaaccaggaagaagttgaaactctgaatagaccaataacaggttctgaaattgaggcaataattaatagcttaccaaccaaaaaaagtccaggaccagatggattcaccgccgaattctaccagaggtacaaggaggacctggtaccattctttctgaaactattccaatcaatagaaaaagagggaatcctccctaactcattttatgaggccagcatcatcctgataccaaagcctggcagagacacaaccaaaaaagagaattttagaccaatatccctgatgaacagtgatacaaaaatcctcaataaaatactggcaaaccgaatccagcagcacatcaaaaagcttatccaccatgatcaagtgggcttcatccctgggatgcaaggctggttcaacatacgcaaatcaataaacataatccagcatataaacagaaccaacgacaaaacccacatgattatctcaatagatgcagaaaaggcctttaacaaaattcaacagcccttcatgctaaaaactctgaataaattaggtattgatggaacctatctcaaaataataagagcaaatttatgacaaacccacagccaatatcatactgaatggacaaaaactggaatcattccctttgaaaactggcacaagacagggatgccctctctcaccactcctattcaacatagtgttggaagttctggccagggcaatcaggcaagagaaagaaataaagggtattcaattaggaaaagaggaagtcaaattgtccctgtttgcagatgacatgattgtatatctagaaaaccccatcgtctcagcccaaaatctccttaagctgataaacaacttcagcaaagtatcaggatacaaaatcaatgtgcaaaaatcacaaatattcttatacaccaataacagacaaacagagagccaaatcatgagtgaactcccattcacaattgcttcaaagacaataaaatacctaggaattcaacttacaagggatgtgaaggacctcttcaaggagaattacaaaccactgctcaatgaaataaaagaagatacaaacaaatggaacaacattccatgctcatgggtaggaagaatcaatatcatgaaaatggccatactgcccaaggtaatttatagattcagtgccatcgccatcaagctaccaatgactttcttcacagaactggaaaaaactactttaaagttcatatggaaccaaaaaagagcccgcattgccaagtcaatcctaagccaaaagaacaaagccggaggcatcatgctacctgacttcaaactatactacaaggctacagtaaccaaaacagcatggtactggtaccaaaacagagatattgatcaatggagcagaacagagccctgagaaagaatgccacatatctacaaccatctgatctttgacaaacctgacaaaaacaagcagtggggaaaggattccctatttaataaatggtgctgggaaaactggctagccatatatagaaagctgaaactggatcccttccttacaccttatacaaaaattaattcaagatggattaaagacttacatgttagacctaaaaccataaaaaccctagaagaaaacctaggcaatatcattcaatacagaggcatgggcaaggacttcatgtctaaaacaccaaaagcaatggcaacaaaagccaaaattgacaaatgggatctaatgaaactaaagagcttctgcacagcaaaagaaactaccatcagagtgaacaggcaaccgacagaatgggagaaaatttttgcaacctactcatctgacaaagggctaatatccagaatctacaatgatctcaaacaaatttacaagaaaaaaacacaaccccatcaacaagtgggggaaggatatgaacagacacttctcaaaagacatttatgcagccaatagacacatgaaaaaatgttcatcatcactggccatcaaagaaatgcaaatcaaaaccacaatgagataccatctcacgccagttagaatggcgatcattaaaaagtcaggaaacaacaggtgctggagaggatgtggagaaaacaggaacacttttacactgttggtgggactgtaaactagttcaaccattgtggaagtcagtgtggtgattcctcagggatctagaactagaaataccatttgacccagccatcccattactgggtatatacccaaaggattataaatcatcctgctataaacacacatgcacacttatgtttattgcagcactattcacaatagcaaagacttggaaccaacccaaatgtccaataatgatagactggattaagaaaatgtggcacatatacaccatggaatgctatgcagccataaaaaatgatgagttcatgtcctttgtagagacatggatgaagctggaaaccatcattctcagcaaactatggcaaggacaaaaaaccaaacactgtatgttctcactcgtaggtgggaattgaacaatgagaacacatggacacaggaaggggaatatcacacactggggcctgttttggggtgggaggagtggggagggatagcattaggagatataccgaatgttaaatgacgagttaatgggtgcagcacaccaacatggcataggtatacatatgtaacaaacctgcacgttgtgtacatgtaccctaaaacttaaagtataaaaaaaaaaattcaaaaacctcagtggcatctaatgagaagcatttattgctcacaagactggatagtgagttctgctgatactgactggactcactctggtctggctatggtctgaggtagcctggccctgggggcgcgatggaggctgactcagctctccccacacctgtctcatgttccagtcaggtagccactggccaagaagccaagctaggaaccagggtatctgactcctgagctaaactctaaccctctacaatactgcctcccaaatataacaccaagtgctaggtacatatcatccacagttttcagacttctgcccaaactgggattctttttagtgtgaagagacctggcctgtggggctgaccctggtgtggctgtgaggcagacacaaagggacatttacatccagtcctgaagattacagtccagccctgaagcaacaactaggaaactattccaaaaggaggggatggggctgagtgtggggttctattctcttcataactttaactagaactcaaattgtgtaccttggtagcatccaatcataaatttattttgtcgtatttgtgatagaaaggaacaagtttatccacaaatttatttatttatttatttatttatttatttatttgagacagggtctgactctacgacccaagctggagggcagtggtgcaatctcagctcactgcaaactctgcctcccaggctcaagccatcctcccgcctctgtctcctgagtagctggaactacaggcacacgccaccacacccagctagtttttgtattttttgtagagatgggttttcaccatgtttcccaagctggtctcaaactcctcaaaagagttaccaagcaggactctgcaaccaataatccttgtgtgaagaggatatttgctcttttccctgtttttctttcttggtacagatgtgtgacctctttttgaaaggtgatagtgactttggtgtattttatttggtggtaatggtcatagccccattaatcacatttcttcccatgagaaagaaaaaccactacatggtcatgctaaggatttcagtccctggggtgaggatggtcttgaatatctcctacattcataactcctccacacatctcagtaggtcactgagcacatcaatggacatgccagttattaaaatacttcacgaatactatgatcatttaccagtatgagttattctctggagcttctaatacttcaatagtactgcatggactcagttgagagttaattcaaaatctcagattatccaattctgtttctttccttccaggcaccacctacctatgatgccgtggtacagatggagtaccttgacatggtggtgaatgaaacactcagattattcccagttgctattagacttgagaggacttgcaagaaagatgttgaaatcaatggggtattcattcccaaagggtcaatggtggtgattccaacttatgctcttcaccatgacccaaagtactggacagagcctgaggagttccgccctgaaaggtacaagtctccagggaaatggagctcaccctgacccaggctggttcaagcatattctgcctctcttaatctacatgacaatcgtgtggttgtacaatcatttgcttgtaagtctttttatcacaaaaaagtgataattatcaaactttacaaaccacagactagaaaaaacgaaactacatccatccacagtcccagcacaagacaaagataatcaattatgtccctgtgggcatttttctacgcctatatagatttttaaaaattagaatggtatcactttttatttggtttgaattgctgcttacttgatttaacaggaaactatccactgacctatattactataaatatacatatatatgtatatatataaatatatatatatgtatatattgcatatgccataaaccatttaaccatgatgttatttcaggtgtataggctttttattcctttctgttttttctatgctgtgccctttagctctctgaatttaacagaaactttaaaacatgcttccacattccatttgctttcaacgttacttgctatttcctctgtagtaattataagagtgcaggctgaggtcctgagaagtcctcatccctaatggtttaagccacttcactgaagacacaagacagcacaggtcctcctggtcctatctgtggctgcagtcctgtgccagctcccttatactctcagtagacatctcacacactcctccttggaggtgtcttgagcatgctcttctgggaattcagggacaaggtcaggccttaggcacagttcgcactctggatatagttggtgttttcccattactgtattattaagcaaaatttagaatgaaatttttagggtactggctggtgattcaggatgcttgggatctagactttcattagcccctacctgcaagtttgctgatgggaggaaccttgtcttgttggtcatggtgtccctagtgctagcatggagtctgcacataatacttgttcacagagtaagtcagagctgaccaagttctctgttttctggagtagaggacttctatgtttcctgcaagctcagcacttccacctcctgtggctgcactaatacgaaatcagagaccactcgctgtacttcactttgaatcactcagtcaccaaaaagatagtgcttgccatgtgtcaggaacttggctaggcagggagaaattcatatgatttatataaatccataaatccatatgatttacataaatccataaattcatgtgatatatacgtatatgtgtgtgtatatatatattagagaatgtttgacatatacacaagtacatgttaccgacaccagcctatagaatagttttcgtgcatctccatatatctatcactggttccaacagccatcaatccatgttagctgccccatccaaatgccaccatcaccctcctcctgactatcatgttattttgaagcaatagcctgtaaatatttcagaatgctctccaaaatataaagactcctgtaaaaacatatgacaacaatgccattattactttctttgaatcaacattttttccttaatataatcaaatatttagaaatcaaatttgaataaaacatgggtcaatcttcaaagaatttatagcttaatggaacagatcaaggaaagcagggatgacactacagtagggtagcatcatatgcccatgtaacttatgtgacttaaactatcctgtaagggtgtgggggagaaagagaggaagagatggagagaagaaaaaggaagagaaggaggaggagaaggaggcagaggagaaggtggacggggaaggtagagaggaggaggaggggaattagaaaaaaagagatgacaggagaaggaaagggaaaaataacaacttgaaatagcacaagacgttttctccttctcctttctcaatgagcatgtgaccaacacaagtgtgagttgaggcaggaatccacttttccatccatcagtcttatcatttatgtgccttttatagtgtgaacacatcaccaccctgaatataattttagtgtttagagataaatattatttgcaacaatattcatctcatctcaagaaacgctcctatagggtatggagaatttaaaggacctgtaggttatgatgattataacgaaataaccaaagcaggatttcaatgaccagcccacaaaagtatcctgtgtactactggttgggaggtggaggggggttgttcttaagtaagaacccctaacatgtaactctgtggtttttatgtttcattaactatttaatctaccaatatggaactaggttcagtaagaagaaggacagcatagatccttacatatacacaccctttggaactggacccagaaactgcattggcatgaggtttgctctcatgaacatgaaacttgctctaatcagagtccttcagaacttctccttcaaaccttgtaaagaaacacaggtcagtacactttctgtatgttttattaagaatttttttaactgaagggtatatattttttaaaagaatatgcatgtttatcttttaataattcattctatgggccaaagaacctacttggatccatctttgatcattaaggatgcttcagttctggacttcaaaacctgtagcattaagaacatcatgtaaagtccacacagattagcatgacatgattatgtgtagtctctttgaacctgagtaagtttaaattcagtttcaagtcaattggaaagaagtgttttgcacaatcatgaagtgcaatgattacctggctgtgacttaaatggtgttctccatcaccagaacctgcagaagctctctcatgacagtggttctcaaccactagctgtatattggaatcaccagggagcttcaaaaattcatgatgcctgtgacatctcagaaattctaaactaattaacccagagcgtgactaggttctgtcatgctgtcgggtgaacccctgattagttctcacgtgaagccaaggtggagaatgactaatttcaggcatttctggtggatatgaaggactaccatagagcagggctatccttactccttgaccttatgttccaggtgatacatttaaagaaagatttagaatcttttctctgaagaagttaaagaacagatgtcattgattcatattaagcaatagcctataagtcttatttccaggaccggtgtatttaatatgcaactctaccccttaagtacactttgtgcttgggagaggaggaggatggagatggttgccatcttatctatggcttcagggcagctgtgtagctttcctatgtgtgtattcaggcagggggctcagccctgagagaaagtgggcctctggcacacctgggacagggaagatattccctggcaagctctcaggcatctcaggctggcacttctttgtatccatggcaatttgctttcccctcactgaactgagatcagaatgttactctgttggtggctcccccaacagtgaaggggtgactcagtgacaatagtgctagaagtatgagtcaaaacactgtacaacttgagaaattccccgtttgcactacgcttggaagccaagaggagatgttaaaaagaaaagaataattctttctgaagacatttcccatcattgcacttgatgggttcaactgggaagggttactagactctggaagttgaaaactgcccacataattaaactgtacaacagctactcaggattaccttgcaagttttaacctataaaaatttaactttatatagcacttccaaaatagtttgccataatacctactaatctggatttaatttttaaaactcatccttttaacttaagatttaaataaaaaaaaaaaaacacgagtccacaagaatttgtctcaggcctggcacagagtcagtgctccataaatattttgttaaacgatggatggtgagtgcttttactatccagtatttacccagcttatagattaagtatgaagagttcaagatacatggtgttaagagtcgtttttatatgcttgcaaagcatttttgtcatattttttctactttgcttccatcttttcttctttcacttcatttattaattctccatatgcttgtttaactattgtagatccccttgaaattagacacgcaaggacttcttcaaccagaaaaacccattgttctaaaggtggattcaagagatggaaccctaagtggagaatgagttattctaaggatttctactttggtcttcaagaaagctgtgccccagaacaccagagatttcaacttagtcaataaaaccttgaaataaagatgggcttaatctaatgtactgcatgagtagttggtgattttgtacattcattgagctctcccagagtctgtgtagagtgttgtgcattatgtagtataaaggaggtgaccaggtaagtgacagataggtagactcagcttctctgcttctcataggactacctctacccacctctagttagcattatcaactcctcctgagctctcatcagagaataaatatttctcaacaatttgatccataacttttaagaaaaataagaattatcatgatgactctaatagtgacatttatatcacgttttatttgtaatattctataagttttatattaagcgaagtgataaaatcccctttacaaaaatattatctgatgccatcctgcacactaaagagaaatctatagaactgaatgactgaaaaccagcaaataaacattttttatcattgtaatcactgttggtgtggggcctttgtcagaattccaatttgattattaacataggtgagagttaatctgctgtgactttgcccattgtttggagaaaatattcatagtttcattctgccttctttgaagaacatattttttgtaacactcaacgaagcacttatcatattattagttatgatttattatttttaccacatctcccctgacatttctggaacacaggaaacatgttttcttatacgtcttgcattccatcttcacctcccaattgtcttaatgcaatgaacactgaataaaaaattgtcaattcgtcagttgattgggcagcatgtctaaaagcactattcattttccttttttattctttcattttccctccttttctgaatactaaagccattaggtgggttgcagccatgtggtagccacacattaaggtggacaagagagtcatggtggctccaagtcagattccaagtgtgctggggaaggcatccacatggaggggcagcctgacctggaagcgggagcccaagcaatcagagaaggggtccacacagaggtgtggccttcaagagcagccagagcctaaatagggcctggagaacccacgtgaggtgaggagggtatccctgagtgggaagggatgggtgagagttggctacatagaagggattgatcacataagtaaataaagtatactggaagctaggtgtgtcacttttgcagaaaagagtcatagattcagaaagggagaaagctagcattaatcctatggtgttagattggaatggatgtatcagtgtacattcatacttttctagatagatagatggatagatagacagatgatagatagataatagatagttgatagataattagatgtaaatatatgtgtttgtatgtgtgagcatgcatgtgtgtgtgtgtgtgtgtgtgtgtgtgtgtgtgtgtgtgtataaaacatatattccctacttcactgatagggctagataacaatgacatttcaatagcaatgagtatacttagtgcctagatcttggcttatgaatatcattttccactgaagggaaccagagctcgttagagaaatagctgatgccagggctaggactaaaaatgttcaagatgagcccaggacactttttgtgccaggaagtaagaaaacactcaaatggtttctaaatggtatttggaaatgatttctaaatgatatttccatatgacttccaaatgatattttaaaaacctaaagactccaccaaagaactattagaactgataaacaaattcagtaaatttgcaggatacaaaatcaacgtacaaaattggtagcacttctatatgccagcaaggaacaatctgaaataaaatcaatagccacaaataaaattaaatacctaggaattaacttaatcaaagaagtgaaagatctctacaatgaaaactataaaacaccgatgaaagaaattgaagaggacacaaaaacatagaaagatattccatgtttatatatttcaagaatcaatattgttaaaaatgcccacaatacccaaagc

(19) United States
(12) Patent Application Publication
(10) Pub. No.: US 2014/0221284 A1
(43) Pub. Date: Aug. 7, 2014

(54) Biomarkers for Rapid Determination of Drug Efficacy

(71) Applicant: Andreas Pfuetzner, Mainz (DE)

(72) Inventor: Andreas Pfuetzner, Mainz (DE)

(73) Assignee: IR2DX, Inc., Morago, CA (US)

(21) Appl. No.: 13/980,813

(22) PCT Filed: Jan. 23, 2012
(86) PCT No.: PCT/US12/22271

§ 371 Date: Jan. 24, 2014

Related U.S. Application Data
(60) Provisional application No. 61/435,155, filed on Jan. 21, 2011.
(60) Provisional application No. 61/435,162, filed on Jan. 21, 2011.

(51) Int. Cl. G01N 33/74; G01N 33/68

(52) U.S. Cl. CPC G01N 33/74; G01N 33/6893
USPC 514/5.9; 506/18; 506/9; 514/7.2; 514/369; 514/312; 514/263.36; 514/428; 514/471

(57) ABSTRACT

The invention provides compositions and methods for determining GLP-1 analog and/or DPPIV inhibitor response in a subject. In one embodiment, the composition comprises a solid support comprising probes for measuring a biomarker panel comprising, for example, adiponectin, C-peptide, hsCRP, insulin, proinsulin. The simultaneous use of multiple biomarkers with independent classification power will increase the performance of the biomarker panel in characterizing GLP-1 analog and DPPIV inhibitor response. The invention also provides methods of treating a subject (e.g. one experiencing cardiodiabetes) and determining the efficacy of a therapy through assaying the various biomarkers of a biomarker panel disclosed herein.

### PRIORITY

This application claims priority under 35 USC §119(e) to U.S. provisional applications U.S. Ser. No. 61/435,155 and U.S. Ser. No. 61/435,162, the contents of both of which are incorporated by reference in their entirety.

### TECHNICAL FIELD

The invention provides compositions and methods for determining a response of a subject to an administered drug, for example a glucagon like peptide-1 (GLP-1) analog or a Dipeptidyl peptidase-4 (DPPIV) inhibitor. The response can be identified by means of a biomarker panel. The simultaneous use of multiple markers (e.g. adiponectin, C-peptide, GLP-1, hsCRP, insulin and proinsulin) with independent classification power and an algorithm to combine, adjust, weigh and interpret the results in a single index will increase the performance of the panel in identifying a response of a subject to a drug.

### BACKGROUND

Not all patients who are treated with a drug respond to such therapy. Currently, identification of responding and nonresponding patients is based on observed clinical efficacy of a drug, but this occurs only after a delay, sometimes of at least several weeks. Further, therapeutic decisions are not easily made at this point because of a number of confounding factors that can influence the clinical outcome, such as noncompliance or incomplete compliance with drug uptake.

Thus there is a need for a means of determining the response of a subject to a drug, in this instance a GLP-1 analog or a DPPIV inhibitor. Such means is provided by various embodiments of the invention described herein.

### BRIEF DESCRIPTION OF DRAWINGS

FIG. 1 shows examples of two different assay configurations.

### DESCRIPTION OF EMBODIMENTS

### Overview

The invention is generally directed to quickly determining the efficacy of therapy using panels of biomarkers. In many cases, for example in the treatment of diabetes and cardiodiabetes, a drug may need to be administered for months before it can be determined whether the drug is working on the particular patient, e.g. whether the patient is responding to the drug treatment, that is whether the patient is a “responder”. The present invention is directed to quickly determining drug efficacy, for example, within a number of days, by taking “before” and “after” measurements of levels of a biomarker panel. That is, a panel of biomarkers as described below can be measured for a patient prior to initiation of administration a drug, including GLP-1 analogs and DPPIV inhibitors (although as described below, the patient may be on other drugs as well at the “start point”). The patient is then started on the drug, and within a few days, as described below, for example at the third day, the panel of biomarkers is measured again. Changes in the quantity of individual markers is an indication of the efficacy of the drug treatment, allowing the physician to then decide to stop the drug treatment, adjust the dose, or add additional drugs from different drug classes.

The panel of biomarkers can comprise a number of different markers, as described below. In addition, as will be appreciated by those in the art and as described below, the level of any individual marker may increase, decrease or stay the same, and the correlation of efficacy with changes is different for each marker. That is, for one marker an increase may indicate responsiveness to the drug, while for a different marker a decrease indicates responsiveness. In addition, in a panel of biomarkers, the levels of markers need not all change to determine if a patient is responding to treatment.

Thus, the present disclosure provides compositions and methods for detecting and measuring the response of subject with cardiodiabetes to a drug through the use of biomarker panels. A biomarker panel that includes adiponectin, C-peptide, high sensitivity C-reactive protein (hsCRP), insulin and proinsulin is particularly useful for detecting a response of a subject with cardiodiabetes to a drug.

A biomarker panel that includes adiponectin, C-peptide, hsCRP, insulin and proinsulin is particularly useful for detecting and measuring a response of a subject with cardiodiabetes administered a GLP-1 analog.

The inclusion of GLP-1 to a biomarker panel that includes adiponectin, C-peptide, hsCRP, insulin and proinsulin is particularly useful for detecting and measuring a response of a subject with cardiodiabetes administered a DPPIV inhibitor. These responses can be detected within a few days of initial drug administration, a time period that is much shorter than the current delay that is on the order of weeks.

Thus, assays for a GLP-1 analog response involving the measurement of adiponectin, C-peptide, hsCRP, insulin and proinsulin have greater value in determining a GLP-1 analog response than those involving any of these biomarkers alone.

Thus, assays for a DPPIV inhibitor response involving the measurement of adiponectin, C-peptide, GLP-1, hsCRP, insulin and proinsulin have greater value in determining DPPIV inhibitor response than those involving any of these biomarkers alone.

The biomarker panels disclosed herein allow the practitioner to directly identify whether a GLP-1 analog or a DPPIV inhibitor is providing a neutral, beneficial or harmful effect. Using the panels disclosed herein, the effects of a GLP-1 analog or a DPPIV inhibitor can be determined in a few (e.g., 1-3) days. Measurements of the biomarkers of any panel disclosed herein may be converted, using a suitable algorithm, to an index, such as a GLP-1 analog response index or a DPPIV inhibitor response index.

The invention also provides for selection of efficient risk-reducing treatment and therapy. The invention provides biomarkers that in various combinations can be used in methods to monitor subjects that are undergoing therapy. Indications of a GLP-1 analog response or a DPPIV inhibitor response allow a caregiver to select or modify therapies or interventions for treating subjects. A number of GLP-1 analog and DPPIV inhibitors have been developed and are available on the market. The biomarkers disclosed herein allow for determining a subject's level of response to a GLP-1 analog, a DPPIV inhibitor or other drug described herein, and for monitoring the effectiveness of drug treatment.

A biomarker panel disclosed herein may be combined with measurements of other biomarkers and clinical parameters to assess a response to a GLP-1 analog and/or a DPPIV inhibitor response.

### Biomarkers

A large number of biomarkers are known for a variety of metabolic, diabetic and cardiovascular conditions. See US/2008/0057590, incorporated by reference in its entirety. Biomarkers may originate from epidemiological studies, animal studies, pathophysiological considerations and end-organ experiments. Ideally, a biomarker will have a high predictive value for a meaningful outcome measure, can be or is validated in appropriately designed prospective trials, reflects therapeutic success by corresponding changes in the surrogate marker results, and should be easy to assess in clinical practice.

The term “surrogate marker,” “biomolecular marker,” “biomarker” or “marker” (also sometimes referred to herein as a “target analyte,” “target species” or “target sequence”) refers to a molecule whose measurement provides information as to the state of a subject. In various exemplary embodiments, the biomarker is used to assess a pathological state. Measurements of the biomarker may be used alone or combined with other data obtained regarding a subject in order to determine the state of the subject. In one embodiment, the biomarker is “differentially present” in a sample taken from a subject of one phenotypic status (e.g., having a disease) as compared with another phenotypic status (e.g., not having the disease). In one embodiment, the biomarker is “differentially present” in a sample taken from a subject undergoing no therapy or one type of therapy as compared with another type of therapy. Alternatively, the biomarker may be “differentially present” even if there is no phenotypic difference, e.g. the biomarkers may allow the detection of asymptomatic risk. A biomarker may be determined to be “differentially present” in a variety of ways, for example, between different phenotypic statuses if the mean or median level or concentration (particularly the expression level of the associated mRNAs as described below) of the biomarker in the different groups is calculated to be statistically significant. Common tests for statistical significance include, among others, t-test, ANOVA, Kruskal-Wallis, Wilcoxon, Mann-Whitney and odds ratio.

As described herein, a biomarker may be, for example, a small molecule, an analyte or target analyte, a lipid (including glycolipids), a carbohydrate, a nucleic acid, a protein, any derivative thereof or a combination of these molecules, with proteins and nucleic acids finding particular use in the invention. As will be appreciated by those in the art, a large number of analytes may be detected using the present methods; basically, any biomarker for which a binding ligand, described below, may be made may be detected using the methods of the invention.

In various embodiments, the biomarkers used in the panels of the invention can be detected either as proteins (i.e., polypeptides) or as nucleic acids (e.g. mRNA or cDNA transcripts) in any combination. In various embodiments, the protein form of a biomarker is measured. As will be appreciated by those in the art, protein assays may be done using standard techniques such as ELISA assays. In various embodiments, the nucleic acid form of a biomarker (e.g., the corresponding mRNA) is measured. In various exemplary embodiments, one or more biomarkers from a particular panel are measured using a protein assay and one or more biomarkers from the same panel are measured using a nucleic acid assay.

Measurements of a biomarker panel comprising or consisting of adiponectin, C-peptide, GLP-1, hsCRP, insulin and proinsulin in various combinations may be used to improve the sensitivity and/or specificity of a diagnostic test compared to a test involving any one of these biomarkers alone.

### Adiponectin

In various embodiments, adiponectin is used as a biomarker. Adiponectin values are useful as a predictive biomarker for insulin resistance and as a monitoring tool in the treatment of insulin resistance related disorders. Full-length adiponectin (f-Ad) is a 30 kDa serum protein specifically secreted by adipocytes. Adiponectin typically circulates in human blood at concentrations ranging between 5 and 12 mg/L, thus accounting for approximately 0.01% of total plasma protein. Schondorf et al., Clin. Lab., 2005, 51: 489-494. Adiponectin concentrations have higher median values in females (about 8.7 mg/L) than in males (about 5.5 mg/L), and may be affected by age as well. Adiponectin concentrations correlate negatively with BMI, visceral fat mass and insulin concentrations. Accordingly, adiponectin is decreased in obese subjects and in patients suffering from type 2 diabetes, macroangiopathy or other metabolic disorders. The lowest adiponectin values have been found in obese patients with both type 2 diabetes and coronary heart disease.

A number of compounds have been shown to affect adiponectin concentrations in a subject. Pfützner et al., Diabetes, Stoffwechsel and Herz, 2007, 16: 91-97 have shown that sulfonylurea, metformin, thiazolidinedione, metformin+sulfonylurea, metformin+thiazolidinedione, sulfonylurea+thiazolidinedione, and metformin+sulfonylurea+thiazolidinedione may have an effect on adiponectin concentrations. Thus, in one embodiment, any of these compounds or combinations may be administered to a subject.

In various embodiments, adiponectin is derived from a peptide sequence according to RefSeq Accession Record NP_{—}004788 or is derived from a nucleic acid sequence according to RefSeq Accession Record NM_{—}004797.

In exemplary embodiments, a protein form of adiponectin is measured. Accordingly, suitable capture binding ligands, as further discussed herein, for the detection or quantification of adiponectin include, but are not limited to, antibodies that are selective for adiponectin.

In various embodiments, a nucleic acid (e.g. mRNA) form of adiponectin is measured. A wide variety of methods for detecting mRNA are known in the art, particularly on arrays. This includes the direct measurement of mRNA as well as treating the same with reverse transcriptase and measuring cDNA levels. Accordingly, suitable capture probes, as further discussed below, for the detection and/or quantification of adiponectin mRNA include, but are not limited to, fragments of the complements of the mRNA sequences of adiponectin. That is, if the mRNA is to be directly detected, a complementary sequence will be used to bind the single stranded mRNA. In general, as for all the capture probes outlined herein, the probes generally are between about 5 and about 100 nucleotides in length, with from about 6 to about 30, about 8 to about 28, and about 16 to about 26 being of particular use in some embodiments.

In some embodiments, the sample (e.g. blood, serum or plasma) levels of adiponectin will increase if a subject is responding to a therapy, such as administration of a disease-modulating drug, as described below. In general, this increase is normally on the level of about 10% to about 100% or more from a reference value. In some embodiments, this increase is about 10% to about 80%, about 20% to about 70%, about 30% to about 60% or about 40% to about 50% from a reference value. In some embodiments, this increase is about 10% to about 20%, about 10% to about 30%, about 10% to about 40%, about 10% to about 50%, about 10% to about 60%, about 10% to about 70%, about 10% to about 90% from a reference value. In preferred embodiments the change is an increase of from a reference value of 50% or more. Thus, in some embodiments, this increase is about 50% to about 100%, about 50% to about 110%, about 50% to about 120%, about 50% to about 130%, about 50% to about 140% or about 50% to about 150% from a reference value. In some embodiments, an increase of at least about a percentage selected from 10%, 20%, 30%, 40%, 50%, 60%, 70%, 80%, 90%, 100%, 110%, 120%, 130%, 140%, 150%, 160%, 170%, 180%, 190% and 200% from a reference value occurs. In some embodiments, an increase of about 10% to about 40% from a reference value occurs. In exemplary embodiments, an increase of at least about 20% from a reference value occurs. Generally, a sample (such as blood or blood component such as plasma or serum) concentration of >10 mg/L indicates a low risk for arteriosclerosis, insulin resistance and other complications; 7-10 mg/L a medium risk and <7 mg/L a high risk. Thus, in one embodiment, a change before and after therapy to a value above 10 mg/L for adiponectin is considered to be indicative of therapy response. In exemplary embodiments, a subject is responding to a therapy if the subject's level of adiponectin in a second sample compared to a first sample changes such that the risk level associated with adiponectin moves from one risk level to a lower risk level, e.g., from high risk to medium risk, high risk to low risk, or medium risk to low risk. Thus, in some embodiments, an increase occurs from a level of about <7 mg/L (high risk) to a level of about 7-10 mg/L (medium risk) or to a level of about >10 mg/L (low risk). In some embodiments, an increase occurs from a level of about 7-10 mg/L (medium risk) to a level of about >10 mg/L (low risk).

It is also possible that the patient is responding to a therapy as shown by changes in other biomarkers, but the levels of adiponectin are not changing in a significant way, since adiponectin suppression reflects the activity of the visceral adipose tissue, which may not be affected by the selected intervention.

### C-Peptide

In various embodiments, C-peptide is used as a biomarker. C-peptide is the middle segment of proinsulin that is between the N-terminal B-chain and the C-terminal A-chain. At physiological concentrations, human C-peptide stimulates glucose transport in a dose-dependent manner and partly shares a common pathway with insulin in stimulating skeletal muscle glucose transportation. C-peptide does not alter the binding of insulin to the insulin receptor nor does it specifically bind to muscle crude membranes. C-peptide stimulates glucose transport by a mechanism independent of insulin receptor and tyrosine kinase activity and in contrast to insulin, catecholamines do not have a counter-regulatory effect on C-peptide mediated glucose transport.

In various embodiments, C-peptide is derived from a peptide sequence according to RefSeq Accession Record NP_{—}000198, particularly the fragment between the N-terminal B-chain and the C-terminal A-chain, or is derived from a nucleic acid sequence according to RefSeq Accession Record NM_{—}000207, particularly the sequence encoding the fragment between the N-terminal B-chain and the C-terminal A-chain. In exemplary embodiments, C-peptide is derived from a peptide sequence according to PDB Accession Record 1T0C_A. In exemplary embodiments, C-peptide has the sequence EAEDLQVGQVELGGGPGAGSLQPLALEGSLQ (SEQ ID NO:1).

In exemplary embodiments, a protein form of C-peptide is measured. Accordingly, suitable capture binding ligands, as further discussed herein, for detection or quantification of C-peptide include, but are not limited to, antibodies that are selective for C-peptide.

In various embodiments, a nucleic acid (e.g. mRNA) form of C-peptide is measured. A wide variety of methods for detecting mRNA are known in the art, particularly on arrays. This includes the direct measurement of mRNA as well as treating the same with reverse transcriptase and measuring cDNA levels. Accordingly, suitable capture probes, as further discussed below, for the detection and/or quantification of C-peptide mRNA include, but are not limited to, fragments of the complements of the mRNA sequences of C-peptide. That is, if the mRNA is to be directly detected, a complementary sequence will be used to bind the single stranded mRNA. In general, as for all the capture probes outlined herein, the probes generally are between about 5 and about 100 nucleotides in length, with from about 6 to about 30, about 8 to about 28, and about 16 to about 26 being of particular use in some embodiments.

In some embodiments, the sample (e.g. blood, serum or plasma) levels of C-peptide will decrease if a subject is responding to a therapy, such as administration of a disease-modulating drug, as described below. In some embodiments, this decrease is about 10% to about 80%, about 20% to about 70%, about 30% to about 60% or about 40% to about 50% from a reference value. In some embodiments, this decrease is about 10% to about 20%, about 10% to about 30%, about 10% to about 40%, about 10% to about 50%, about 10% to about 60%, about 10% to about 70%, or about 10% to about 90% from a reference value. In some embodiments, a decrease of at least about a percentage selected from 10%, 11%, 12%, 13%, 14%, 15%, 16%, 17%, 18%, 19%, 20%, 30%, 40%, 50%, 60%, 70%, 80%, 90%, 95%, 96%, 97%, 98% and 99% from a reference value will occur. In some embodiments, a decrease of about 10% to about 40% from a reference value occurs. In some embodiments, a decrease of at least about 15% from a reference value occurs. In preferred embodiments a decrease of at least 10% from a reference value is observed. More preferably, in some embodiments a decrease of at 20-30% from a reference value is observed. In some embodiments, levels of C-peptide decrease from an initial level to below a level selected from about 400 pmol/L, about 500 pmol/L, about 600 pmol/L, about 700 pmol/L and about 800 pmol/L, preferably below about 600 pmol/L.

In some embodiments, a response to a therapy will cause C-peptide to change from an initial level to a level either above or below a reference value, such as a level selected from about 400 pmol/L, about 500 pmol/L, about 600 pmol/L, about 700 pmol/L and about 800 pmol/L, preferably about 600 pmol/L. C-peptide (fasting) concentration is greater than about 600 pmol/L in an individual with a high risk for disease and is less than about 600 pmol/L in an individual with a low risk for disease.

It is also possible that the patient is responding to a therapy as shown by changes in other biomarkers, but the levels of C-peptide are not changing in a significant way.

High Sensitivity C-Reactive Protein (hsCRP)

In various embodiments, CRP (C-reactive protein) or hsCRP (high sensitivity C-reactive protein) is used as a biomarker. CRP is a member of the pentraxin family, comprising five noncovalently associated protomers arranged symmetrically around a central pore and has a molecular weight of 118,000 Da. (See generally, halal et al., Hypertension, 2004, 44: 6-11) CRP is a marker of inflammation that has been shown to predict incident myocardial infarction, stroke, peripheral arterial disease, and sudden cardiac death process. Ridker, Circulation, 2003, 107: 363-369. Various epidemiological studies involving individuals with no prior history of cardiovascular disease have shown that a single, non-fasting measure of CRP is a strong predictor of future vascular events. The predictive value of CRP has proven independent of major traditional risk factors, such as age, smoking, cholesterol levels, blood pressure and diabetes.

High-sensitivity CRP assays have been developed and are now widely available. (Roberts et al., Clinical Chemistry 2001, 47: 444-450.) In one embodiment, a biomarker, such as hsCRP, is measured by immune turbidometry.

In various embodiments, hsCRP is derived from a peptide sequence according to RefSeq Accession Record NP_{—}000558 or is derived from a nucleic acid sequence according to RefSeq Accession Record NM_{—}000567.

In exemplary embodiments, a protein form of hsCRP is measured. Accordingly, suitable capture binding ligands, as further discussed herein, for detection or quantification of hsCRP include, but are not limited to, antibodies that are selective for hsCRP.

In various embodiments, a nucleic acid (e.g. mRNA) form of hsCRP is measured. A wide variety of methods for detecting mRNA are known in the art, particularly on arrays. This includes the direct measurement of mRNA as well as treating the same with reverse transcriptase and measuring cDNA levels. Accordingly, suitable capture probes, as further discussed below, for the detection and/or quantification of hsCRP mRNA include, but are not limited to, fragments of the complements of the mRNA sequences of hsCRP. That is, if the mRNA is to be directly detected, a complementary sequence will be used to bind the single stranded mRNA. In general, as for all the capture probes outlined herein, the probes generally are between about 5 and about 100 nucleotides in length, with from about 6 to about 30, about 8 to about 28, and about 16 to about 26 being of particular use in some embodiments.

In some embodiments, the sample (e.g. blood, serum or plasma) levels of hsCRP will decrease if a subject is responding to a therapy, such as administration of a disease-modulating drug, as described below. In some embodiments, this decrease is about 10% to about 80%, about 20% to about 70%, about 30% to about 60% or about 40% to about 50% from a reference value. In some embodiments, this decrease is about 10% to about 20%, about 10% to about 30%, about 10% to about 40%, about 10% to about 50%, about 10% to about 60%, about 10% to about 70%, about 10% to about 80%, or about 10% to about 90% from a reference value. In some embodiments, a decrease of at least about a percentage selected from 10%, 11%, 12%, 13%, 14%, 15%, 16%, 17%, 18%, 19%, 20%, 30%, 40%, 50%, 60%, 70%, 80%, 90%, 95%, 96%, 97%, 98% and 99% from a reference value will occur. In some embodiments, a decrease of about 10% to about 40% from a reference value occurs. In some embodiments, a decrease of at least about 15% from a reference value occurs. In some embodiments, a decrease of about 25% to about 30% from a reference value indicates a response. A decrease of about 10% to about 100% can also be observed. In preferred embodiments the change is a decrease of at least 50% or more from a reference value. More preferably, a decrease of at least about 55%, 60%, 65%, 70%, 75%, 80%, 85%, 90%, 95% from a reference value will occur.

In patients with chronic systemic inflammation, decreases occur from levels of about 3-10 mg/L to levels of about 2-3 mg/L, with changes of at least about 10-15% to about 30-40% being more determinative of a response.

An hsCRP level after two weeks of treatment between 0 to 1 mg/L indicates a low remaining systemic inflammation and corresponding cardiovascular risk, 1 to 3 mg/L indicates a moderate remaining risk and 3 to 10 mg/L indicates a high remaining risk.

Values above 10 mg/L may be caused by other unspecific inflammation (e.g. infections) and do not have a predictive value for cardiovascular risk. In exemplary embodiments, a subject is responding to a therapy if the subject's level of hsCRP in a second sample compared to a first sample changes such that the risk level associated with hsCRP moves from one risk level to a lower risk level, e.g., from high risk to medium risk, high risk to low risk, or medium risk to low risk. Thus, in some embodiments, a decrease occurs from a level of about 3-10 mg/L to a level of about 1-3 mg/L or to a level of about 0-1 mg/L. In some embodiments, a decrease occurs from a level of about 1-3 mg/L to a level of about 0-1 mg/L.

It is also possible that the patient is responding to a therapy, such as a disease-modulating drug as shown by changes in other biomarkers, but the levels of hsCRP are not changing in a significant way. This is specifically the case if other risk factors or diseases interfere with chronic systemic inflammation in regards to macrophage activation.

### Insulin

In various embodiments, insulin is used as a biomarker. Insulin is a peptide hormone having about 51 amino acid residues and a molecular weight of 5.8 kDa. The hormone is a member of a larger family of molecules that all have some degree of homology in their sequence, for example, the insulin-like growth factors (IGF-I and IGF-II). In some instances, insulin can undergo glycation.

In an exemplary embodiment, insulin is derived from a peptide sequence according to RefSeq Accession Record NP_{—}000198, particularly wherein the sequence corresponding to C-peptide has been deleted and wherein the A and B chains are bound together by disulfide bonds, or is derived from a nucleic acid sequence according to RefSeq Accession Record NM_{—}000207.

In exemplary embodiments, a protein form of insulin is measured. Accordingly, suitable capture binding ligands, as further discussed herein, for detection and/or quantification of insulin include, but are not limited to, antibodies that are selective for insulin.

In various embodiments, a nucleic acid (e.g. mRNA) form of insulin is measured. A wide variety of methods for detecting mRNA are known in the art, particularly on arrays. This includes the direct measurement of mRNA as well as treating the same with reverse transcriptase and measuring cDNA levels. Accordingly, suitable capture probes, as further discussed below, for the detection and/or quantification of insulin mRNA include, but are not limited to, fragments of the complements of the mRNA sequences of insulin. That is, if the mRNA is to be directly detected, a complementary sequence will be used to bind the single stranded mRNA. In general, as for all the capture probes outlined herein, the probes generally are between about 5 and about 100 nucleotides in length, with from about 6 to about 30, about 8 to about 28, and about 16 to about 26 being of particular use in some embodiments.

In some embodiments, the sample (e.g. blood, serum or plasma) levels of insulin will decrease if a subject is responding to a therapy, such as administration of a disease-modulating drug, as described below. In some embodiments, this decrease is about 10% to about 80%, about 20% to about 70%, about 30% to about 60% or about 40% to about 50% from a reference value. In some embodiments, this decrease is about 10% to about 20%, about 10% to about 30%, about 10% to about 40%, about 10% to about 50%, about 10% to about 60%, about 10% to about 70%, or about 10% to about 90% from a reference value. In some embodiments, a decrease of at least about a percentage selected from 10%, 11%, 12%, 13%, 14%, 15%, 16%, 17%, 18%, 19%, 20%, 30%, 40%, 50%, 60%, 70%, 80%, 90%, 95%, 96%, 97%, 98% and 99% from a reference value will occur. In some embodiments, a decrease of about 10% to about 40% from a reference value occurs. In some embodiments, a decrease of at least about 15% from a reference value occurs. In preferred embodiments, a decrease of at least 10% from a reference value occurs. More preferably, in some embodiments a decrease of at least 20% or at 30% from a reference value occurs. In some embodiments, sample levels of insulin decrease from an initial level to below a level selected from about 15 mU/L, about 20 mU/L, about 25 mU/L, about 30 mU/L and about 35 mU/L, preferably below about 25 mU/L. In exemplary embodiments, a response to a therapy will cause insulin to change from an initial level to a level either above or below a reference value, such as a level selected from about 15 mU/L, about 20 mU/L, about 25 mU/L, about 30 mU/L and about 35 mU/L, preferably about 25 mU/L. The fasting insulin concentration is greater than about 25 mU/L in an individual with a high risk for disease and less than about 25 mU/L in an individual with a low risk for disease.

It is also possible that the patient is responding to a therapy, such as a disease modulating drug, as shown by changes in other biomarkers, but the levels of insulin are not changing in a significant way.

### Proinsulin

In various embodiments, intact proinsulin is used as a biomarker. As used herein, “proinsulin” refers to the prohormone precursor to insulin made in the β-cell of the islets of Langerhans. Proinsulin may be cleaved within β-cell granules to result in two separate molecules: C-peptide and insulin. Partial processing of proinsulin may result in split or “des” forms of proinsulin. (Clark, Ann Clin Biochem, 1999, 36: 541-564.) The term “proinsulin” as used herein preferably refers to the unprocessed form of proinsulin, that is, “intact proinsulin” and so these terms can be used interchangeably.

Intact proinsulin concentrations are related to atherosclerosis and cardiovascular disease. If the demand for insulin triggered by insulin resistance reaches a certain threshold, insufficient cleavage capacity of β-cell carboxypeptidase H leads to an increased secretion of intact proinsulin in addition to the desired insulin molecule. Intact proinsulin, however, has been demonstrated to be an independent cardiovascular risk factor. Assessment of β-cell function by determination of intact proinsulin facilitates the selection of the most promising therapy and also serves to monitor treatment success in the further course of the disease. Intact proinsulin may serve as a marker to investigate β-cell function and allows for a secretion-oriented staging of type 2 diabetes.

Chemiluminescence is one technique that can be used to measure intact proinsulin and other biomarkers. Two types of chemiluminescence assays are able to specifically measure uncleaved “intact” proinsulin and “total” proinsulin (proinsulin and its specific and non-specific degradation products) in human plasma (Invitron Intact Proinsulin and Invitron Total Proinsulin; Invitron Ltd, Monmouth, UK). Other methods suitable for proinsulin include without limitation chromatography, particularly HPLC, stable isotope dilution mass spectrometry assays, and ELISA. See, generally, Clark.

In an exemplary embodiment, intact proinsulin is derived from a peptide sequence according to RefSeq Accession Record NP_{—}000198, particularly a fragment of the sequence described therein in which the signal peptide has been cleaved, or is derived from a nucleic acid sequence according to RefSeq Accession Record NM_{—}000207, particularly a fragment of the sequence described therein coding for a protein in which the signal peptide has been cleaved.

In exemplary embodiments, a protein form of intact proinsulin is measured. Accordingly, suitable capture binding ligands, as further discussed herein, for detection or quantification of intact proinsulin include, but are not limited to, antibodies that are selective for intact proinsulin.

In various embodiments, a nucleic acid (e.g. mRNA) form of intact proinsulin is measured. A wide variety of methods for detecting mRNA are known in the art, particularly on arrays. This includes the direct measurement of mRNA as well as treating the same with reverse transcriptase and measuring cDNA levels. Accordingly, suitable capture probes, as further discussed below, for the detection and/or quantification of intact proinsulin mRNA include, but are not limited to, fragments of the complements of the mRNA sequences of intact proinsulin. That is, if the mRNA is to be directly detected, a complementary sequence will be used to bind the single stranded mRNA. In general, as for all the capture probes outlined herein, the probes generally are between about 5 and about 100 nucleotides in length, with from about 6 to about 30, about 8 to about 28, and about 16 to about 26 being of particular use in some embodiments.

In some embodiments, the sample (e.g. blood, serum or plasma) levels of intact proinsulin will decrease if a subject is responding to a therapy, such as administration of a disease-modulating drug, as described below. In some embodiments, this decrease is about 10% to about 80%, about 20% to about 70%, about 30% to about 60% or about 40% to about 50% from a reference value. In some embodiments, this decrease is about 10% to about 20%, about 10% to about 30%, about 10% to about 40%, about 10% to about 50%, about 10% to about 60%, about 10% to about 70%, about 10% to about 80%, or about 10% to about 90% from a reference value. In some embodiments, a decrease of at least about a percentage selected from 10%, 11%, 12%, 13%, 14%, 15%, 16%, 17%, 18%, 19%, 20%, 30%, 40%, 50%, 60%, 70%, 80%, 90%, 95%, 96%, 97%, 98% and 99% from a reference value will occur. In some embodiments, a decrease of about 10% to about 40% from a reference value occurs. In some embodiments, a decrease of at least about 15% from a reference value occurs. In preferred embodiments, a decrease of at least 10% from a reference value occurs. More preferably, in some embodiments a decrease of at least 20% or at 30% from a reference value occurs. For example, a decrease from 10-30% from a reference value in a patient would evidence that the patient is responding to a therapy. In some patients with severe β-cell dysfunction, this decrease is normally from a level of about 12-20 pmol/L to a level of about 6-8 pmol/L, with a value below about 11 pmol/L being the therapeutic target level. Generally, an intact proinsulin concentration above 11 pmol/L indicates a high risk of (3-cell dysfunction while a concentration at or below 11 pmol/L indicates a low risk. In exemplary embodiments, a subject is responding to a therapy if the subject's level of intact proinsulin in a second sample compared to a first sample changes such that the risk level associated with intact proinsulin moves from one risk level to a lower risk level, e.g., from high risk to medium risk, high risk to low risk, or medium risk to low risk.

In some embodiments, sample levels of intact proinsulin decrease from an initial level to below a level selected from about 9 pmol/L, about 10 pmol/L, about 11 pmol/L, about 12 pmol/L and about 13 pmol/L, preferably below about 11 pmol/L.

In some embodiments, a response to a therapy will cause intact proinsulin to change from an initial level to a level either above or below a reference value, such as a level selected from about 9 pmol/L, about 10 pmol/L, about 11 pmol/L, about 12 pmol/L and about 13 pmol/L, preferably about 11 pmol/L.

It is also possible that the patient is responding to a therapy, such as a disease modulating drug, as shown by changes in other biomarkers, but the levels of intact proinsulin are not changing in a significant way. In this case, however, a significant cardiovascular risk still remains.

### Glucagon-Like Peptide 1 (GLP-1)

In this section the use of glucagon-like peptide 1 (GLP-1) as a biomarker is described and is different from the use of GLP-1 as a drug, described below. GLP-1 is an incretin released from L-cells in the small intestine. Peripheral administration of GLP-1 has been shown to decrease food intake. GLP-1 is also a fragment of proglucagon, which itself is a 160 amino acid fragment of preproglucagon. See Holst et al., Physiological Reviews, 2007, 87: 1409-1439. The predominant biologically active form of GLP-1 in humans is GLP-1(7-36)-NH₂, which corresponds to proglucagon (78-107). GLP-1(7-36)-NH₂ is rapidly inactivated in the body. For example, dipeptidyl peptidase IV (DPP IV) degrades GLP-1(7-36)-NH₂ to GLP-1(9-36)-NH₂. Enzymes such as neutral endopeptidase (NEP) 24.11 are known to hydrolyze GLP-1(7-36)-NH₂ at positions 15, 16, 18, 19, 20, 27, 28, 31 and 32. Thus, in various embodiments, GLP-1 refers to preproglucagon (for example, according to RefSeq Accession Record NP_{—}002045 or the nucleic acid sequence in RefSeq Accession Record NM_{—}002054) or preferably a fragment thereof, any of which can be derivatized or underivatized. In various embodiments, GLP-1 refers to a derivatized or underivatized fragment of proglucagon. In various exemplary embodiments, GLP-1 refers to, for example, GLP-1(7-36)-NH₂, GLP-1(7-37), GLP-1(9-36)-NH₂, or any fragment resulting from the hydrolysis of GLP-1(7-36)-NH₂ at positions 15, 16, 18, 19, 20, 27, 28, 31, 32 or other positions. In various embodiments, GLP-1 has the sequence HAEGTFTSDVSSYLEGQAAKEFIAWLVKGR, (SEQ ID NO: 2) wherein the final arginine is optionally amidated. In exemplary embodiments, GLP-1 refers to GLP-1(6-37), which is preferably amidated. Other GLP-1 polypeptides useful in the present invention can be found in Chi et al., Bioorganic and Medicinal Chemistry, 2008, 16: 7607-7614, Holst et al., Physiological Reviews, 2007, 87: 1409-1439 (all incorporated by reference in their entirety for all purposes) and in other references in the art.

In various embodiments, GLP-1 refers to an amidated fragment of preproglucagon or an amidated fragment of proglucagon.

In exemplary embodiments, a polypeptide form of GLP-1 is measured. Accordingly, suitable capture binding ligands, as further discussed below, for detection or quantification of GLP-1 include, but are not limited to, antibodies that are selective for GLP-1.

In some embodiments, a nucleic acid form of GLP-1 (e.g. mRNA derived from a sequence according to RefSeq Accession Record NM_{—}002054) is measured. A wide variety of methods for detecting mRNA are known in the art, particularly on arrays. This includes the direct measurement of mRNA as well as treating the same with reverse transcriptase and measuring cDNA levels. Accordingly, suitable capture probes, as further discussed below, for the detection and/or quantification of GLP-1 mRNA include, but are not limited to, fragments of the complements of the mRNA sequences of GLP-1. That is, if the mRNA is to be directly detected, a complementary sequence will be used to bind the single stranded mRNA. In general, as for all the capture probes outlined herein, the probes generally are between about and about 100 nucleotides in length, with from about 6 to about 30, about 8 to about 28, and about 16 to about 26 being of particular use in some embodiments.

In some embodiments, the sample (e.g. blood, serum or plasma) levels of GLP-1 will decrease if a subject is responding to a therapy, such as administration of a disease-modulating drug, as described below. In some embodiments, this decrease is about 10% to about 80%, about 20% to about 70%, about 30% to about 60% or about 40% to about 50% from a reference value. In some embodiments, this decrease is about 10% to about 20%, about 10% to about 30%, about 10% to about 40%, about 10% to about 50%, about 10% to about 60%, about 10% to about 70%, about 10% to about 80%, or about 10% to about 90% from a reference value. In some embodiments, a decrease of at least about a percentage selected from 10%, 11%, 12%, 13%, 14%, 15%, 16%, 17%, 18%, 19%, 20%, 30%, 40%, 50%, 60%, 70%, 80%, 90%, 95%, 96%, 97%, 98% and 99% from a reference value will occur. In some embodiments, a decrease of about 10% to about 40% from a reference value occurs. In some embodiments, a decrease of at least about 15% from a reference value occurs. In preferred embodiments, a decrease of at least 10% from a reference value occurs. More preferably, in some embodiments a decrease of at least 20% or at least 30% from a reference value occurs. For example, a decrease from 10-30% from a reference value in a patient would evidence that the patient is responding to a therapy.

It is also possible that the patient is responding to a therapy, such as administration of a disease-modulating drug, as shown by changes in other biomarkers, but the levels of GLP-1 are not changing in a significant way.

In some embodiments, a biomarker panel that includes adiponectin, C-peptide, GLP-1, hsCRP, insulin, proinsulin and CYP3A5 is useful for determining DPPIV inhibitor response.

### Biomarker Panels

Any combination of the biomarkers described herein can be used to assemble a biomarker panel, which is detected or measured as described herein. As is generally understood in the art, a combination may refer to an entire set or any subset (i.e. subcombination) thereof. According to context, the term combination may mean more than one but fewer than all. The term “biomarker panel,” “biomarker profile,” or “biomarker fingerprint” refers to a set of biomarkers. As used herein, these terms can also refer to any form of the biomarker that is measured. Thus, if adiponectin is part of a biomarker panel, then either an adiponectin polypeptide or an adiponectin mRNA, for example, could be considered to be part of the panel. While individual biomarkers are useful as diagnostics, it has been found that a combination of biomarkers can sometimes provide greater value in determining a particular status than single biomarkers alone. Specifically, the detection of a plurality of biomarkers in a sample can increase the sensitivity and/or specificity of the test. Thus, in various embodiments, a biomarker panel may include 2, 3, 4, 5, 6, 7, 8, 9, 10 or more types of biomarkers. In various exemplary embodiments, the biomarker panel consists of a minimum number of biomarkers to generate a maximum amount of information. Thus, in various embodiments, the biomarker panel consists of 2, 3, 4, 5, 6, 7, 8, 9 or 10 types of biomarkers. Where a biomarker panel “consists of” a set of biomarkers, no biomarkers other than those of the set are present.

The present invention provides a biomarker panel comprising or consisting of any combination of the biomarkers outlined herein.

In various exemplary embodiments, the biomarker panel comprises additional biomarkers. Such additional biomarkers may, for example, increase the specificity and/or sensitivity the test. For example, additional biomarkers may be those that are currently evaluated in the clinical laboratory and used in traditional global risk assessment algorithms, such as those from the San Antonio Heart Study, the Framingham Heart Study, and the National Cholesterol Education Program Expert Panel on Detection, Evaluation, and Treatment of High Blood Cholesterol in Adults (Adult Treatment Panel III), also known as NCEP/ATP III. Additional biomarkers suitable for biomarker panels include, without limitation and if not already selected, any combination of biomarkers selected from adiponectin, angiotensin II, complement factor 3, leptin, mRNAx, NFκB, IL-6, MMP-9, TNFα, NFκB, eNOS, PPARγ, MCP-1, PAI-1, ICAM/VCAM, E-selectin, P-selectin, von Willebrand factor, sCD40L, insulin, proinsulin, glucose, HbAlc, lipids such as free fatty acids, total cholesterol, triglycerides, VLDL, LDL, small dense LDL, oxidized LDL, resistin, HDL, NO, IκB-a, IκB-β, p105, RelA, MIF, inflammatory cytokines, molecules involved in signaling pathways, traditional laboratory risk factors and any biomarkers disclosed in US/2008/0057590. It should be understood that in these embodiments, the biomarker panel can include any combination of biomarkers selected from adiponectin, C-peptide, GLP-1, hsCRP, insulin, proinsulin and the remainder of these markers.

For example, MMP-9 and/or MCP-1 can be included within the biomarker panel. A 10%, 20%, or 30% decrease in MMP-9 would evidence that the patient is responding to a therapy. A 10%, 20%, or 30% decrease in MCP-1 would evidence that the patient is responding to a therapy.

Glucose as used herein includes, without limitation, fasting glucose as well as glucose concentrations taken during and after the oral glucose tolerance test, such as 120 minute Glucose. Insulin as used herein includes, without limitation, fasting insulin and insulin concentrations taken during and after the oral glucose tolerance test, such as 120 minute Insulin.

Traditional laboratory risk factors are also understood to encompass without limitation, fibrinogen, lipoprotein (a), c-reactive protein (including hsCRP), D-dimer, and homocysteine.

A biomarker can also be a clinical parameter, although in some embodiments, the biomarker is not included in the definition of “biomarker”. The term “clinical parameter” refers to all non-sample or non-analyte biomarkers of subject health status or other characteristics, such as, without limitation, age, ethnicity, gender, diastolic blood pressure and systolic blood pressure, family history, height, weight, waist and hip circumference, body-mass index, as well as others such as Type I or Type II Diabetes Mellitus or Gestational Diabetes Mellitus (collectively referred to here as Diabetes), resting heart rate, homeostatic model assessment (HOMA), HOMA insulin resistance (HOMA-IR), intravenous glucose tolerance (SI(IVGT)), β-cell function, macrovascular function, microvascular function, atherogenic index, blood pressure, low-density lipoprotein/high-density lipoprotein ratio, intima-media thickness, and UKPDS risk score. Other clinical parameters are disclosed in US/2008/0057590.

### Panels of Particular Use

A. Panels for Determining or Measuring a Response to a GLP-1 Analog

In various exemplary embodiments, the biomarker panel comprises adiponectin, C-peptide, hsCRP, insulin and proinsulin. In various exemplary embodiments, the biomarker panel comprises any combination of adiponectin, C-peptide, hsCRP, insulin and proinsulin. In various exemplary embodiments, the biomarker panel consists of adiponectin, C-peptide, hsCRP, insulin and proinsulin. In various exemplary embodiments, the biomarker panel consists of any combination of adiponectin, C-peptide, hsCRP, insulin and proinsulin.

In various exemplary embodiments, the biomarker panel comprises or consists of adiponectin, C-peptide, hsCRP, insulin, proinsulin and 1, 2, 3, 4 or more additional biomarkers. In various exemplary embodiments, the biomarker panel comprises or consists of any combination of adiponectin, C-peptide, hsCRP, insulin, proinsulin and 1, 2, 3, 4 or more additional biomarkers.

B. Panels for Determining or Measuring a Response to a DPPIV Inhibitor

In various exemplary embodiments, the biomarker panel comprises adiponectin, C-peptide, GLP-1, hsCRP, insulin and proinsulin. In various exemplary embodiments, the biomarker panel comprises any combination of adiponectin, C-peptide, GLP-1, hsCRP, insulin and proinsulin. In various exemplary embodiments, the biomarker panel consists of adiponectin, C-peptide, GLP-1, hsCRP, insulin and proinsulin. In various exemplary embodiments, the biomarker panel consists of any combination of adiponectin, C-peptide, GLP-1, hsCRP, insulin and proinsulin.

In various exemplary embodiments, the biomarker panel comprises or consists of adiponectin, C-peptide, GLP-1, hsCRP, insulin, proinsulin and 1, 2, 3, 4 or more additional biomarkers. In various exemplary embodiments, the biomarker panel comprises or consists of any combination of adiponectin, C-peptide, GLP-1, hsCRP, insulin, proinsulin and 1, 2, 3, 4 or more additional biomarkers.

In various exemplary embodiments, the biomarker panel comprises adiponectin, C-peptide, GLP-1, hsCRP, insulin, proinsulin and CYP3A, and in preferred embodiments CYP3A5. In various exemplary embodiments, the biomarker panel comprises any combination of adiponectin, C-peptide, GLP-1, hsCRP, insulin, proinsulin and CYP3A5. In various exemplary embodiments, the biomarker panel consists of adiponectin, C-peptide, GLP-1, hsCRP, insulin, proinsulin and CYP3A5. In various exemplary embodiments, the biomarker panel consists of any combination of adiponectin, C-peptide, GLP-1, hsCRP, insulin, proinsulin and CYP3A5.

In various exemplary embodiments, the biomarker panel comprises or consists of adiponectin, C-peptide, GLP-1, hsCRP, insulin, proinsulin, CYP3A5 and 1, 2, 3, 4 or more additional biomarkers. In various exemplary embodiments, the biomarker panel comprises or consists of any combination of adiponectin, C-peptide, GLP-1, hsCRP, insulin, proinsulin, CYP3A5 and 1, 2, 3, 4 or more additional biomarkers.

### Measurement and Detection of Biomarkers

Using any of the methods and compositions described herein, a sample can be assayed to determine concentrations of a biomarker panel. Thus, in one aspect, the invention provides a method of assaying a sample comprising taking a measurement of a biomarker panel in the sample. In one aspect, the invention provides a method of acquiring data relating to a sample comprising taking a measurement of a biomarker panel in the sample. In one aspect, the invention provides a method of measuring analyte concentrations in a sample comprising taking a measurement of a biomarker panel in the sample. Any method or use herein could comprise contacting a sample with a solid support comprising a capture binding ligand or capture probe for each biomarker of a biomarker panel and taking a measurement of the biomarker panel (e.g. to determine biomarker concentrations). Any biomarker panel disclosed herein can be used in these and other methods and uses.

Different types of biomarkers and their measurements can be combined in the compositions and methods of the present invention. In various embodiments, the protein form of the biomarkers is measured. In various embodiments, the nucleic acid form of the biomarkers is measured. In exemplary embodiments, the nucleic acid form is mRNA. In various embodiments, measurements of protein biomarkers are used in conjunction with measurements of nucleic acid biomarkers.

Biomarkers generally can be measured and detected through a variety of assays, methods and detection systems known to one of skill in the art. The term “measuring,” “detecting,” or “taking a measurement” refers to a quantitative or qualitative determination of a property or characteristic of an entity, e.g., quantifying the amount or the activity level of a molecule. The term “concentration” or “level” can refer to an absolute or relative quantity. Measuring a molecule may also include determining the absence or presence of the molecule. A measurement may refer to one observation under a set of conditions or an equally- or differently-weighted average of a plurality of observations under the same set of conditions. Thus, in various embodiments, a measurement of the concentration of a biomarker is derived from one observation of the concentration, and in various embodiments, a measurement of a biomarker is derived from an equally- or differently-weighted average of a plurality of observations of the concentration. In various embodiments, measuring a biomarker panel comprises measuring the concentrations of each member of the biomarker panel in a sample.

Various methods include but are not limited to refractive index spectroscopy (RI), ultra-violet spectroscopy (UV), fluorescence analysis, radiochemical analysis, near-infrared spectroscopy (near-IR), infrared (IR) spectroscopy, nuclear magnetic resonance spectroscopy (NMR), light scattering analysis (LS), mass spectrometry, pyrolysis mass spectrometry, nephelometry, dispersive Raman spectroscopy, gas chromatography, liquid chromatography, gas chromatography combined with mass spectrometry, liquid chromatography combined with mass spectrometry, matrix-assisted laser desorption ionization-time of flight (MALDI-TOF) combined with mass spectrometry, ion spray spectroscopy combined with mass spectrometry, capillary electrophoresis, colorimetry and surface plasmon resonance (such as according to systems provided by Biacore Life Sciences). See also WO/2004/056456 and WO/2004/088309. In this regard, biomarkers can be measured using the above-mentioned detection methods, or other methods known to the skilled artisan. Other biomarkers can be similarly detected using reagents that are specifically designed or tailored to detect them.

Protein Assays

As will be appreciated by those in the art, there are a large number of possible proteinaceous target analytes and target species that may be detected using the present invention. The term “protein,” “polypeptide” or “oligopeptide” (used interchangeably herein) refers to at least two or more peptides or amino acids joined by one or more peptide bonds. A protein or an amino acid may be naturally or nonnaturally occurring and may be also be an analog, a derivative or a peptidomimetic structure. A protein can have a wild-type sequence, a variant of a wild-type sequence or either of these containing one or more analogs or derivatized amino acids. A variant may contain one or more additions, deletions or substitutions of one or more peptides compared to wild-type or a different variant sequence. Examples of derivatized amino acids include, without limitation, those that have been modified by the attachment of labels (described below); acetylation; acylation; ADP-ribosylation; amidation; covalent attachment of flavin, a heme moiety, a nucleotide, a lipid or phosphatidylinositol; cross-linking; cyclization; disulfide bond formation; demethylation; esterification; formation of covalent crosslinks, cystine or pyroglutamate; formylation; gamma carboxylation; glycosylation; GPI anchor formation; hydroxylation; iodination; methylation; myristoylation; oxidation; proteolytic processing; phosphorylation; prenylation; racemization; selenoylation; sulfation; and ubiquitination. Such modifications are well-known to those of skill in the art and have been described in great detail in the scientific literature. Several particularly common modifications such as glycosylation, lipid attachment, sulfation, gamma-carboxylation, hydroxylation and ADP-ribosylation, for instance, are described in basic texts, such as Creighton, Proteins—Structure and Molecular Properties, 2d ed. (New York: W. H. Freeman and Company, 1993). Many detailed reviews are available on this subject, such as in Johnson, ed., Posttranslational Covalent Modification of Proteins (New York: Academic Press, 1983); Seifter et al., Meth. Enzymol., 1990, 182: 626-646; and Rattan et al., Ann. N.Y. Acad. Sci., 1992, 663: 48-62. As discussed below, when the protein is used as a binding ligand, it may be desirable to utilize protein analogs to retard degradation by sample contaminants.

Thus, in many embodiments, the invention provides a solid support comprising or consisting of one or more capture ligands selective for a protein form of one or more members of a biomarker panel. In one aspect, the invention provides methods of assaying a sample comprising contacting the sample with a solid support comprising one or more capture ligands, each selective for a different biomarker of a biomarker panel, and measuring each of the biomarkers of the biomarker panel. In various embodiments, a capture ligand is referred to as a capture binding ligand, which can be, for example, an antibody.

The term “solid support” or “substrate” refers to any material that can be modified to contain discrete individual sites appropriate for the attachment or association of a capture binding ligand (or capture probe, described below). Suitable substrates include metal surfaces such as gold, electrodes, glass and modified or functionalized glass, plastics (including acrylics, polystyrene and copolymers of styrene and other materials, polypropylene, polyethylene, polybutylene, polycarbonate, polyurethanes, Teflon, derivatives thereof, etc.), polysaccharides, nylon or nitrocellulose, resins, mica, silica or silica-based materials including silicon and modified silicon, carbon, metals, inorganic glasses, fiberglass, ceramics, GETEK (a blend of polypropylene oxide and fiberglass) and a variety of other polymers. Of particular use in the present invention are the ClonDiag™ materials described below.

As discussed herein, the capture binding ligands can be covalently attached to the surface, for example using capture binding ligands terminally modified with functional groups, for example amino groups, that are attached to modified surfaces such as silanized glass. Alternatively, non-covalent attachment, such as electrostatic, hydrophobic/hydrophilic adhesion can be utilized. As is appreciated by those in the art and discussed herein, a large number of attachments are possible on a wide variety of surfaces.

The measurement of biomarkers in the present invention can be accomplished using biochip assays. By “biochip” or “chip” herein is meant a composition generally comprising a solid support or substrate to which a capture ligand (or capture probe or capture biding probe, described below) is attached. Generally, where a biochip is used for measurements of protein and nucleic acid biomarkers, the protein biomarkers are measured on a chip separate from that used to measure the nucleic acid biomarkers. Of particular interest for the measurement of biomarkers in the present invention are biochip assays. By “biochip” or “chip” herein is meant a composition generally comprising a solid support or substrate to which a capture ligand (also called an adsorbent, affinity reagent or binding ligand, or when nucleic acid is measured, a capture probe) is attached and can bind either proteins, nucleic acids or both. Generally, where a biochip is used for measurements of protein and nucleic acid biomarkers, the protein biomarkers are measured on a chip separate from that used to measure the nucleic acid biomarkers. For nonlimiting examples of additional platforms and methods useful for measuring nucleic acids, see US/2006/0275782, US/2005/0064469 and DE10201463. In various embodiments, biomarkers are measured on the same platform, such as on one chip. In various embodiments, biomarkers are measured using different platforms and/or different experimental runs.

A number of different biochip array platforms as known in the art may be used. For example, the compositions and methods of the present invention can be implemented with array platforms such as GeneChip (Affymetrix), CodeLink Bioarray (Amersham), Expression Array System (Applied Biosystems), SurePrint microarrays (Agilent), Sentrix LD BeadChip or Sentrix Array Matrix (Illumina) and Verigene (Nanosphere).

In various embodiments, biomarkers are measured on the same platform, such as on one chip. In various embodiments, biomarkers are measured using different platforms and/or different experimental runs.

Frequently, the surface of a biochip comprises a plurality of addressable locations, each of which comprises a capture binding ligand (or capture probe, described below). An “array location,” “addressable location,” “pad” or “site” herein means a location on the substrate that comprises a covalently attached capture binding ligand. An “array” herein means a plurality of capture binding ligands (or capture probes, described below) in a regular, ordered format, such as a matrix. The size of the array will depend on the composition and end use of the array. Arrays containing from about two or more different capture binding ligands (or capture probes described below) to many thousands can be made. Generally, the array will comprise a plurality of types of capture binding ligands depending on the end use of the array. In the present invention, the array can include controls, replicates of the markers and the like. Exemplary ranges are from about 3 to about 50. In some embodiments, the compositions of the invention may not be in array format; that is, for some embodiments, compositions comprising a single capture ligand may be made as well. In addition, in some arrays, multiple substrates may be used, either of different or identical compositions. Thus for example, large arrays may comprise a plurality of smaller substrates.

In various embodiments, where a target species is a protein, the ArrayTube™ biochip comprises capture binding ligands such as antibodies. A sample is contacted with the biochip, and any target species present in the sample is allowed to bind to the capture binding ligand antibodies. A soluble capture binding ligand or a detection compound such as a horseradish peroxidase conjugated antibody is allowed to bind to the target species. A dye, such as TMB, is then added and allowed to react with the horseradish peroxidase, causing precipitation and a color change that is detected by a suitable detection device. Further description of protein detection using ArrayTube™ is found in, for example, Huelseweh B, Ehricht R and Marschall H-J, Proteomics, 2006, 6, 2972-2981; and ClonDiag, ArrayTube (AT) Experiment Guideline for Protein-Based Applications, version 1.2, 2007, all incorporated by reference in their entirety.

By “binding ligand,” “capture binding ligand,” “capture binding species,” or “capture ligand” herein is meant a compound that is used to detect the presence of or to quantify, relatively or absolutely, a target analyte or target species (used interchangeably) and that will bind to the target analyte or target species. Generally, the capture binding ligand allows the attachment of a target analyte or target species to a solid support for the purposes of detection as further described herein. Attachment of the target species to the capture binding ligand may be direct or indirect. In exemplary embodiments, the target species is a biomarker. As will be appreciated by those in the art, the composition of the binding ligand will depend on the composition of the biomarker. Binding ligands for a wide variety of biomarkers are known or can be readily found using known techniques. For example, when the biomarker is a protein, the binding ligands include proteins (particularly including antibodies or fragments thereof (FAbs, etc.) as discussed further below) or small molecules. The binding ligand may also have cross-reactivity with proteins of other species. Antigen-antibody pairs, receptor-ligands, and carbohydrates and their binding partners are also suitable analyte-binding ligand pairs.

Capture binding ligands that are useful in the present invention may be “selective” for, “specifically bind” or “selectively bind” their target, such as a protein. A ligand that is “selective for,” “specifically binds” or “selectively binds” a biomarker means that the ligand binds the biomarker with specificity sufficient to differentiate between the biomarker and other components or contaminants of the sample. Typically, specific or selective binding can be distinguished from non-specific or non-selective binding when the dissociation constant (K_{D}) is less than about 1×10⁻⁵ M, less than about 1×10⁻⁶ M or less than about 1×10⁻⁷ M. Specific binding can be detected, for example, by ELISA, immunoprecipitation, coprecipitation, with or without chemical crosslinking, two-hybrid assays and the like. Appropriate controls can be used to distinguish between “specific” and “non-specific” binding.

Nucleic Acid Assays

In various exemplary embodiments, the biomarker is a nucleic acid. The term “nucleic acid,” “oligonucleotide” or “polynucleotide” herein means at least two nucleotides covalently linked together. A nucleic acid of the present invention will generally contain phosphodiester bonds, although in some cases, for example in the use of binding ligand probes, nucleic acid analogs are included that may have alternate backbones, comprising, for example, phosphoramide (Beaucage et al., Tetrahedron, 49(10): 1925 (1993) and references therein; Letsinger, J. Org. Chem. 35: 3800 (1970); Sprinzl et al., Eur. J. Biochem. 81:579 (1977); Letsinger et al., Nucl. Acids Res. 14: 3487 (1986); Sawai et al, Chem. Lett. 13(5): 805 (1984); Letsinger et al., J. Am. Chem. Soc. 110:4470 (1988); and Pauwels et al., Chemica Scripta 26:141 (1986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphosphoroamidite linkages (see Eckstein, Oligonucleotides and Analogues: A Practical Approach, (Oxford University Press, 1991), and peptide nucleic acid backbones and linkages (see Egholm, J. Am. Chem. Soc. 114: 1895 (1992); Meier et al., Chem. Int. Ed. Engl. 31: 1008 (1992); Nielsen, Nature, 365: 566 (1993); Carlsson et al., Nature, 380: 207 (1996), all of which are incorporated by reference). Other analog nucleic acids include those with positive backbones (Denpcy et al., Proc. Natl. Acad. Sci. USA 92: 6097 (1995)), non-ionic backbones (U.S. Pat. Nos. 5,386,023; 5,637,684; 5,602,240; 5,216,141 and 4,469,863; Kiedrowshi et al., Angew. Chem. Intl. Ed. English 30: 423 (1991); Letsinger et al., J. Am. Chem. Soc. 110: 4470 (1988); Letsinger et al., Nucleoside & Nucleotide 13: 1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4: 395 (1994); Jeffs et al., J. Biomolecular NMR 34: 17 (1994); and Horn et al., Tetrahedron Lett. 37: 743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev., 24: 169-176 (1995)). Several nucleic acid analogs are described in Rawls, C & E News, 35 (Jun. 2, 1997). All of these references are hereby expressly incorporated by reference. These modifications of the ribose-phosphate backbone may be done to increase the stability and half-life of such molecules in physiological environments. As will be appreciated by those in the art, all of these nucleic acid analogs may find use in the present invention. In addition, mixtures of naturally occurring nucleic acids and analogs can be made.

In various embodiments, variants of the sequences described herein, including proteins and nucleic acids based on e.g. splice variants, variants comprising a deletion, addition, substitution, fragment, preproprotein, processed preproprotein (e.g. without a signaling peptide), processed proprotein (e.g. resulting in an active form), nonhuman sequences and variant nonhuman sequences may be used as biomarkers. In some embodiments, the variant sequence has a homology compared to a parent sequence, such as a sequence described herein, of about a percentage selected from 30%, 40%, 50%, 60%, 70%, 80%, 90%, 91%, 92%, 93%, 94%, 95%, 96%, 97%, 98% and 99%.

Detecting or measuring the concentration (e.g. to determine transcription level) of a biomarker involves binding of the biomarker to a “capture probe” when the nucleic acid form (e.g. mRNA) of the biomarker is to be detected on a solid support. In that sense, the biomarker is a target sequence. The term “target sequence” or “target nucleic acid” herein means a nucleic acid sequence that may be a portion of a gene, a regulatory sequence, genomic DNA, cDNA, RNA including mRNA and rRNA, or others. As is outlined herein, the target sequence may be a target sequence found directly in a sample. The target sequence may in some embodiments be a secondary target such as a product of an amplification reaction such as PCR etc. In some embodiments, measuring a nucleic acid can thus refer to measuring the complement of the nucleic acid. It may be any length, with the understanding that longer sequences are more specific.

Capture probes that “selectively bind” (i.e., are “complementary” or “substantially complementary”) to or are “selective for” a target nucleic acid find use in the present invention. “Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T (or A and U), or C and G. Two single stranded RNA or DNA molecules may be said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least about 14 to about 25 nucleotides, preferably at least about 75%, more preferably at least about 90% complementary. See, generally, M. Kanehisa, Nucleic Acids Res., 2004, 12: 203.

“Duplex” means at least two oligonucleotides and/or polynucleotides that are fully or partially complementary undergo Watson-Crick type base pairing among all or most of their nucleotides so that a stable complex is formed. The terms “annealing” and “hybridization” are used interchangeably to mean the formation of a stable duplex. In one embodiment, stable duplex means that a duplex structure is not destroyed by a stringent wash, e.g. conditions including temperature of about 5° C. less that the Tₘ of a strand of the duplex and low monovalent salt concentration, e.g. less than 0.2 M, or less than 0.1 M. “Perfectly matched” in reference to a duplex means that the poly- or oligonucleotide strands making up the duplex form a double stranded structure with one another such that every nucleotide in each strand undergoes Watson-Crick basepairing with a nucleotide in the other strand. The term “duplex” includes the pairing of nucleoside analogs, such as deoxyinosine, nucleosides with 2-aminopurine bases, PNAs, and the like, that may be employed. A “mismatch” in a duplex between two oligonucleotides or polynucleotides means that a pair of nucleotides in the duplex fails to undergo Watson-Crick bonding.

Using sequence information provided by the database entries for the biomarker sequences, expression of the biomarker sequences can be detected (if present) and measured using known techniques. For example, sequences in sequence database entries or sequences disclosed herein can be used to construct probes for detecting biomarker RNA sequences in, e.g., Northern blot hybridization analyses or methods which specifically, and, preferably, quantitatively amplify specific nucleic acid sequences. As another example, the sequences can be used to construct primers for specifically amplifying the biomarker sequences in, e.g., amplification-based detection methods such as reverse-transcription based polymerase chain reaction (RT-PCR). When alterations in gene expression are associated with gene amplification, deletion, polymorphisms and mutations, sequence comparisons in test and reference populations can be made by comparing relative amounts of the examined DNA sequences in the test and reference cell populations. In addition to Northern blot and RT-PCR, RNA can also be measured using, for example, other target amplification methods (e.g., transcription-mediated amplification (TMA), strand displacement amplification (SDA), nucleic acid sequence based amplification (NASBA) and real time PCR), signal amplification methods (e.g., bDNA), nuclease protection assays, in situ hybridization and the like.

In one aspect, the invention provides a probe set comprising or consisting of a plurality of probes for detecting a biomarker panel.

Thus, in one embodiment, a probe set for detecting or measuring a response to GLP-1 analogs comprises or consists of a plurality of probes for detecting adiponectin, C-peptide, hsCRP, insulin, and proinsulin. In one embodiment, a probe set for detecting or measuring a response to GLP-1 analogs comprises or consists of a capture binding ligand (or capture binding probe) selective for adiponectin, a capture binding ligand (or capture binding probe) selective for C-peptide capture binding ligand selective for hsCRP, a capture binding ligand (or capture binding probe) selective for insulin, and a capture binding ligand (or capture binding probe) selective for proinsulin.

In one embodiment, a probe set for detecting or measuring a response to DPPIV inhibitors comprises or consists of a plurality of probes for detecting adiponectin, C-peptide, GLP-1, hsCRP, insulin, proinsulin and optionally CYP3A5. In one embodiment, a probe set comprises or consists of a capture binding ligand (or capture binding probe) selective for adiponectin, a capture binding ligand (or capture binding probe) selective for C-peptide, a capture binding ligand (or capture binding probe) selective for GLP-1, a capture binding ligand (or capture binding probe) selective for hsCRP, a capture binding ligand (or capture binding probe) selective for insulin, and a capture binding ligand (or capture binding probe) selective for proinsulin.

The target sequence may also comprise different target domains; for example, a first target domain of the sample target sequence may hybridize to a first capture probe, a second target domain may hybridize to a label probe (e.g. a “sandwich assay” format), etc. The target domains may be adjacent or separated as indicated. Unless specified, the terms “first” and “second” are not meant to confer an orientation of the sequences with respect to the 5′-3′ orientation of the target sequence. For example, assuming a 5′-3′ orientation of the target sequence, the first target domain may be located either 5′ to the second domain, or 3′ to the second domain.

In some embodiments, a target sequence comprises a detectable label, as described below. In exemplary embodiments, the label is added to the target sequence during amplification of the target through the use of either labeled primers or labeled dNTPs, both of which are well known in the art.

In some embodiments, the primers or dNTPs are labeled with biotin, which can then be contacted with a streptavidin/label complex. In one embodiment, the streptavidin/label complex comprises a fluorophore. In an exemplary embodiment, the streptavidin/label complex comprises an enzymatic label. For example, the enzymatic label can be horseradish peroxidase, and upon contact with a precipitating agent, such as 3,3′,5,5′-tetramethylbenzidine (TMB) or o-dianisidine (3,3′-dimethoxybenzidine (dihydrochloride), Fast Blue B), an optically detectable precipitation reaction occurs. This has a particular benefit in that the optics for detection do not require the use of a fluorimeter or other detector, which can add to the expense of carrying out the methods.

In one aspect, the invention provides a composition comprising a solid support comprising one or more capture probes which bind a nucleic acid, each selective for a different biomarker of a biomarker panel. In various embodiments, the composition further comprises a soluble binding probe for one or more biomarkers of a biomarker panel. In one aspect, the invention provides methods of assaying a sample comprising contacting the sample with a solid support comprising one or more capture probe, each selective for a different biomarker of a biomarker panel, and measuring each of the biomarkers of the biomarker panel.

Combination Assays

Different types of biomarkers and their measurements can be combined in the compositions and methods of the present invention. In various embodiments, the protein form of the biomarkers is measured. In various embodiments, the nucleic acid form of the biomarkers is measured. In exemplary embodiments, the nucleic acid form is mRNA. In various embodiments, measurements of protein biomarkers are used in conjunction with measurements of nucleic acid biomarkers.

In various embodiments, the binding ligand may be nucleic acid. Nucleic acid binding ligands find particular use when proteins are the targets; alternatively, as is generally described in U.S. Pat. Nos. 5,270,163; 5,475,096; 5,567,588; 5,595,877; 5,637,459; 5,683,867; 5,705,337 and related patents, hereby incorporated by reference, nucleic acid “aptamers” can be developed for binding to virtually any biomarker. Nucleic acid binding ligands also find particular use when nucleic acids are binding targets. There is a wide body of literature relating to the development of binding partners based on combinatorial chemistry methods. In these embodiments, when the binding ligand is a nucleic acid, preferred compositions and techniques are outlined in WO/1998/020162, hereby incorporated by reference.

Nucleic acid-nucleic acid binding proteins pairs are also useful. In general, the smaller of the pair is attached to the NTP for incorporation into the primer. Preferred binding partner pairs include, but are not limited to, biotin (or imino-biotin) and streptavidin, digeoxinin and Abs, and Prolinx™ reagents.

### Assay Detection

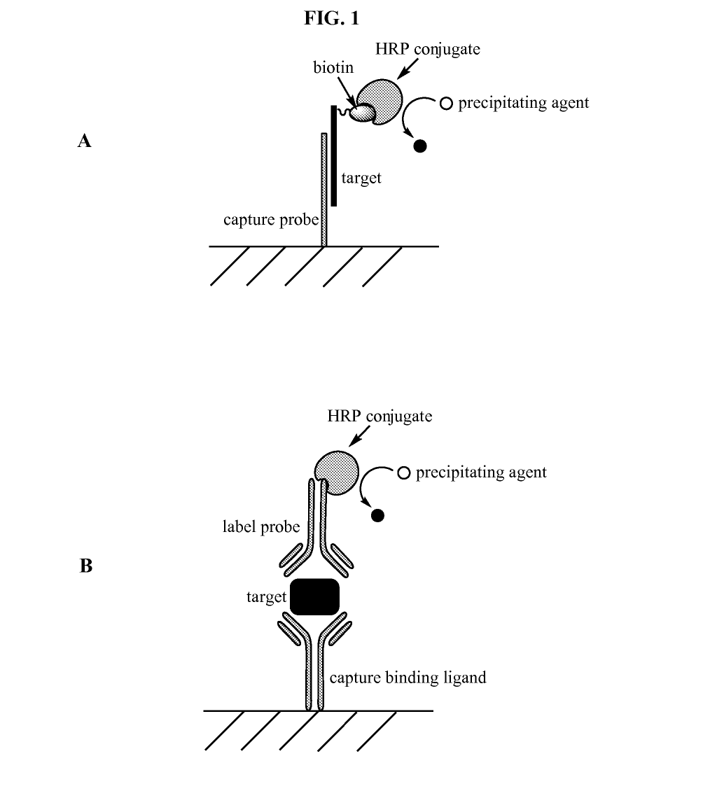

A schematic of example assay configurations that can used for detection is shown in FIGS. 1A and 1B. FIG. 1A shows a configuration that can be used to detect a nucleic acid target. A capture probe is attached to a solid support, and a target labeled with biotin binds to the capture probe. A horseradish peroxidase (HRP) conjugate binds to the biotin, and when a soluble precipitating agent contacts the HRP, a visible precipitate is created. FIG. 1B shows a configuration that can be used to detect a polypeptide target, following a similar principle. In FIG. 1B, the capture binding ligand and label probes are depicted as antibodies. The HRP conjugate can be directly bound to the label probe or via a biotin-streptavidin linkage. These configurations are particularly suited for use with the ClonDiag platform.

In various exemplary embodiments, detection and measurement of biomarkers utilizes colorimetric methods and systems in order to provide an indication of binding of a target analyte or target species. In colorimetric methods, the presence of a bound target species such as a biomarker will result in a change in the absorbance or transmission of light by a sample or substrate at one or more wavelengths. Detection of the absorbance or transmission of light at such wavelengths thus provides an indication of the presence of the target species.

A detection system for colorimetric methods includes any device that can be used to measure colorimetric properties as discussed above. Generally, the device is a spectrophotometer, a colorimeter or any device that measures absorbance or transmission of light at one or more wavelengths. In various embodiments, the detection system comprises a light source; a wavelength filter or monochromator; a sample container such as a cuvette or a reaction vial; a detector, such as a photoresistor, that registers transmitted light; and a display or imaging element. In some embodiments, a change in the colorimetric properties of a sample can be detected directly by the naked eye, i.e., by direct visual inspection.

In embodiments finding particular use herein, a sandwich format is utilized, in which target species are unlabeled. In these embodiments, a “capture” or “anchor” binding ligand is attached to the detection surface as described herein, and a soluble binding ligand (which can be referred to as a “signaling probe,” “label probe” or “soluble capture ligand”) binds independently to the target species and either directly or indirectly comprises at least one label or detectable marker. In these embodiments, the label probe can comprise either a primary (e.g. a fluorophore) or a secondary (biotin or enzyme) label. In the sandwich formats of the invention, an enzyme can serve as the secondary label, bound to the soluble capture ligand. In some cases, the soluble capture ligand comprises biotin, which is then bound to a enzyme-streptavidin (e.g. comprising horseradish peroxidase) complex and forms a colored precipitate with the addition of a precipitating agent, such as 3,3′,5,5′-tetramethylbenzidine (TMB), o-dianisidine (3,3′-dimethoxybenzidine (dihydrochloride), Fast Blue B) or the like. In one embodiment, the label probe comprises biotin, and a streptavidin/enzyme complex is used, as discussed herein. In some embodiments, an enzyme such as horseradish peroxidase is directly conjugated to a soluble binding ligand.

As used herein, the term “fluorescent signal generating moiety” or “fluorophore” refers to a molecule or part of a molecule that absorbs energy at one wavelength and re-emits energy at another wavelength. Fluorescent properties that can be measured include fluorescence intensity, fluorescence lifetime, emission spectrum characteristics, energy transfer, and the like.

Signals from single molecules can be generated and detected by a number of detection systems, including, but not limited to, scanning electron microscopy, near field scanning optical microscopy (NSOM), total internal reflection fluorescence microscopy (TIRFM), and the like. Abundant guidance is found in the literature for applying such techniques for analyzing and detecting nanoscale structures on surfaces, as evidenced by the following references that are incorporated by reference: Reimer et al, editors, Scanning Electron Microscopy Physics of Image Formation and Microanalysis, 2nd Edition (Springer, 1998); Nie et al, Anal. Chem., 78: 1528-1534 (2006); Hecht et al, Journal Chemical Physics, 112: 7761-7774 (2000); Zhu et al, editors, Near-Field Optics: Principles and Applications (World Scientific Publishing, Singapore, 1999); Drmanac, WO/2004/076683; Lehr et al, Anal. Chem., 75: 2414-2420 (2003); Neuschafer et al, Biosensors & Bioelectronics, 18: 489-497 (2003); Neuschafer et al, U.S. Pat. No. 6,289,144; and the like.

Thus, a detection system for fluorophores includes any device that can be used to measure fluorescent properties as discussed above. In various embodiments, the detection system comprises an excitation source, a fluorophore, a wavelength filter to isolate emission photons from excitation photons and a detector that registers emission photons and produces a recordable output, in some embodiments as an electrical signal or a photographic image. Examples of detection devices include without limitation spectrofluorometers and microplate readers, fluorescence microscopes, fluorescence scanners (including e.g. microarray readers) and flow cytometers.

In various exemplary embodiments, a ClonDiag chip platform is used for the colorimetric detection of biomarkers. In various embodiments, a ClonDiag ArrayTube™ (AT) is used. One unique feature of the ArrayTube is the combination of a micro probe array (the biochip) and micro reaction vial. In various embodiments, where a target sequence is a nucleic acid, detection of the target sequence is done by amplifying and biotinylating the target sequence contained in a sample and optionally digesting the amplification products. The amplification product is then allowed to hybridize with probes contained on the ClonDiag chip. A solution of a streptavidin-enzyme conjugate, such as Poly horseradish peroxidase (HRP) conjugate solution, is contacted with the ClonDiag chip. After washing, a dye solution such as o-dianisidine substrate solution is contacted with the chip. Oxidation of the dye results in precipitation that can be detected colorimetrically. Further description of the ClonDiag platform is found in Monecke S, Slickers P, Hotzel H et al., Clin Microbiol Infect 2006, 12: 718-728; Monecke S, Berger-Bächi B, Coombs C et al., Clin Microbiol Infect 2007, 13: 236-249; Monecke S, Leube I and Ehricht R, Genome Lett 2003, 2: 106-118; German Patent DE 10201463; US Publication US/2005/0064469 and ClonDiag, ArrayTube (AT) Experiment Guideline for DNA-Based Applications, version 1.2, 2007, all incorporated by reference in their entirety. Use of the ClonDiag platform for genotyping is described in Sachse K et al., BMC Microbiology 2008, 8: 63; Monecke S and Ehricht R, Clin Microbiol Infect 2005, 11: 825-833; and Monecke S et al., Clin Microbiol Infect 2008, 14(6): 534-545. One of skill in the art will appreciate that numerous other dyes that react with a peroxidase can be utilized to produce a colorimetric change, such as 3,3′,5,5′-tetramethylbenzidine (TMB). For information on specific assay protocols, see www.clondiag.com/technologies/publications.php. Such dyes may be referred to as a precipitating agent herein.

Transmission detection and analysis is performed with a ClonDiag AT reader instrument. Suitable reader instruments and detection devices include the ArrayTube Workstation ATS and the ATR 03.

In addition to ArrayTube™, the ClonDiag ArrayStrip™ (AS) can be used. The ArrayStrip™ provides a 96-well format for high volume testing. Each ArrayStrip™ consists of a standard 8-well strip with a microarray integrated into the bottom of each well. Up to 12 ArrayStrips™ can be inserted into one microplate frame enabling the parallel multiparameter testing of up to 96 samples. The ArrayStrip™ can be processed using the ArrayStrip™ Processor ASP, which performs all liquid handling, incubation, and detection steps required in array based analysis. In various embodiments, where a protein is detected, a method of using the ArrayStrip™ to detect the protein comprises conditioning the AS array with buffer or blocking solution; loading of up to 96 sample solutions in the AS wells to allow for binding of the protein; 3× washing; conjugating with a secondary antibody linked to HRP; 3×washing; precipitation staining with TMB; and AS array imaging and optional data storage.

Those skilled in the art will be familiar with numerous additional immunoassay formats and variations thereof which may be useful for carrying out the method disclosed herein. See generally E. Maggio, Enzyme-Immunoassay, (CRC Press, Inc., Boca Raton, Fla., 1980); see also U.S. Pat. Nos. 4,727,022; 4,659,678; 4,376,110; 4,275,149; 4,233,402; and 4,230,767.

In general, immunoassays carried out in accordance with the present invention may be homogeneous assays or heterogeneous assays. In a homogeneous assay the immunological reaction usually involves the specific antibody (e.g., anti-biomarker protein antibody), a labeled analyte, and the sample of interest. The signal arising from the label is modified, directly or indirectly, upon the binding of the antibody to the labeled analyte. Both the immunological reaction and detection of the extent thereof can be carried out in a homogeneous solution. Immunochemical labels which may be employed include free radicals, radioisotopes, fluorescent dyes, enzymes, bacteriophages, or coenzymes.

In a heterogeneous assay approach, the reagents are usually the sample, the antibody, and means for producing a detectable signal. Samples as described above may be used. The antibody can be immobilized on a support, such as a bead (such as protein A and protein G agarose beads), plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the sample. Means for producing a detectable signal include the use of radioactive labels, fluorescent labels, or enzyme labels. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays include immunoblotting, immunofluorescence methods, immunoprecipitation, chemiluminescence methods, electrochemiluminescence (ECL) or enzyme-linked immunoassays.

Antibodies can be conjugated to a solid support suitable for a diagnostic assay (e.g., beads such as protein A or protein G agarose, microspheres, plates, slides or wells formed from materials such as latex or polystyrene) in accordance with known techniques, such as passive binding. Antibodies as described herein may likewise be conjugated to detectable labels or groups such as radiolabels (e.g., ³⁵S, ¹²⁵I, ¹³¹I), enzyme labels (e.g., horseradish peroxidase, alkaline phosphatase), and fluorescent labels (e.g., fluorescein, Alexa, green fluorescent protein, rhodamine) in accordance with known techniques.

As used herein, the term “antibody” means a protein comprising one or more polypeptides substantially encoded by all or part of the recognized immunoglobulin genes. The recognized immunoglobulin genes, for example in humans, include the kappa (K), lambda (λ) and heavy chain genetic loci, which together compose the myriad variable region genes, and the constant region genes mu (μ), delta (δ), gamma (γ), epsilon (ε) and alpha (α), which encode the IgM, IgD, IgG, IgE, and IgA isotypes respectively. Antibody herein is meant to include full length antibodies and antibody fragments, and may refer to a natural antibody from any organism, an engineered antibody or an antibody generated recombinantly for experimental, therapeutic or other purposes as further defined below. Antibody fragments include Fab, Fab′, F(ab′)₂, Fv, scFv or other antigen-binding subsequences of antibodies and can include those produced by the modification of whole antibodies or those synthesized de novo using recombinant DNA technologies. The term “antibody” refers to both monoclonal and polyclonal antibodies. Antibodies can be antagonists, agonists, neutralizing, inhibitory or stimulatory.

Kits

The invention further provides kits for performing any of the methods disclosed herein for a number of medical (including diagnostic and therapeutic), industrial, forensic and research applications. In some embodiments, the kits are for determining therapy response in a subject. Kits may comprise a portable carrier, such as a box, carton, tube or the like, having in close confinement therein one or more containers, such as vials, tubes, ampoules, bottles, pouches, envelopes and the like. In various embodiments, a kit comprises one or more components selected from one or more media or media ingredients and reagents for the measurement of the various biomarkers and biomarker panels disclosed herein. For example, kits of the invention may also comprise, in the same or different containers, in any combination, one or more DNA polymerases, one or more primers, one or more probes, one or more binding ligands, one or more suitable buffers, one or more nucleotides (such as deoxynucleoside triphosphates (dNTPs) and preferably labeled dNTPs), one or more detectable labels and markers and one or more solid supports, any of which is described herein. The components may be contained within the same container, or may be in separate containers to be admixed prior to use. The kits of the present invention may also comprise one or more instructions or protocols for carrying out the methods of the present invention. The kits may comprise a detector for detecting a signal generated through use of the components of the invention in conjunction with a sample. The kits may also comprise a computer or a component of a computer, such as a computer-readable storage medium or device. Examples of storage media include, without limitation, optical disks such as CD, DVD and Blu-ray Discs (BD); magneto-optical disks; magnetic media such as magnetic tape and internal hard disks and removable disks; semi-conductor memory devices such as EPROM, EEPROM and flash memory; and RAM. The computer-readable storage medium may comprise software encoding references to the various therapies and treatment regimens disclosed herein. The software may be interpreted by a computer to provide the practitioner with treatments according to various measured concentrations of biomarkers as provided herein. In various embodiments, the kit comprises a biomarker assay involving a lateral-flow-based point-of-care rapid test with detection of risk thresholds, or a biochip with quantitative assays for the constituent biomarkers. Generally, any of the methods disclosed herein can comprise using any of the kits (comprising primers, probes, labels, ligands, reagents and solid supports in any combination) disclosed herein.

In one aspect, the invention provides a kit comprising a solid support comprising or consisting of one or more capture binding ligands selective for a protein form of one or more members of a biomarker panel. In one aspect, the invention provides a kit comprising a solid support comprising or consisting of one or more capture probes selective for a nucleic acid form of one or more members of a biomarker panel. In one aspect, the invention provides a kit comprising (a) a solid support comprising or consisting of one or more capture binding ligands selective for a protein form of one or more members of a biomarker panel and (b) a solid support comprising or consisting of one or more capture probes selective for a nucleic acid form of one or more members of a biomarker panel.

In one aspect, the invention provides use of a kit comprising a solid support comprising probes selective for members of a biomarker panel for determining a second therapy for a subject that has undergone a first therapy, wherein the subject is suffering from a disease. In one embodiment, the use comprises (a) contacting a first sample from the subject with a solid support of the kit; (b) taking a first measurement of the concentrations of the biomarker panel in the first sample; (c) effecting a first therapy on the subject; (d) contacting a second sample from the subject with the solid support of the kit; (e) taking a second measurement of the concentrations of the biomarker panel in the second sample and (f) making a comparison of the first and second measurements.

In one aspect, the invention provides use of a kit comprising a solid support comprising probes selective for members of a biomarker panel for determining whether a subject belongs to a population that would benefit from a second therapy, wherein the subject has undergone a first therapy. In one embodiment, the use comprises (a) contacting a first sample from the subject with a solid support of the kit; (b) taking a first measurement of the concentrations of the biomarker panel in the first sample; (c) effecting a first therapy on the subject; (d) contacting a second sample from the subject with the solid support of the kit; (e) taking a second measurement of the concentrations of the biomarker panel in the second sample and (f) making a comparison of the first and second measurements.

### Methods of Diagnosing and Treating

The compositions and methods of the present invention can be used in the prognosis, diagnosis and treatment of disease in a subject.

A “subject” in the context of the present invention is an animal, preferably a mammal. The mammal can be a human, non-human primate, mouse, rat, dog, cat, horse, or cow, but are not limited to these examples. In various exemplary embodiments, a subject is human and may be referred to as a “patient”. Mammals other than humans can be advantageously used as subjects that represent animal models of a disease or for veterinarian applications. A subject can be one who has been previously diagnosed or identified as having a disease, and optionally has already undergone, or is undergoing, a therapeutic intervention for a disease. Alternatively, a subject can also be one who has not been previously diagnosed as having a disease. For example, a subject can be one who exhibits one or more risk factors for a disease, or one who does not exhibit a disease risk factor, or one who is asymptomatic for a disease. A subject can also be one who is suffering from or at risk of developing a disease. In certain embodiments, the subject can be already undergoing therapy or can be a candidate for therapy.

The invention provides compositions and methods for laboratory and point-of-care tests for measuring biomarkers in a sample from a subject. The invention can be generally applied for a number of different diseases. In exemplary embodiments, the disease is insulin resistance. In exemplary embodiments, the disease is cardiovascular disease or risk. In exemplary embodiments, the disease is atherosclerosis. In exemplary embodiments, the disease is diabetes mellitus. In exemplary embodiments, the disease is obesity. In exemplary embodiments, the disease is cardiodiabetes.

“Cardiodiabetes” refers to patients with insulin resistance and β-cell dysfunction without elevation of blood glucose who are not identified as suffering from diabetes mellitus. These normoglycemic patients, however, experience the same elevated cardiovascular risk, which is predominantly linked to vascular insulin resistance. A cardiodiabetic subject might not exhibit one or more of the normal symptoms of type 2 diabetes including, but not limited to, hyperglycemia, fatigue, weight gain, excessive eating, poor wound healing and infections. A cardiodiabetic subject is at high risk for cardiovascular disease and may experience events such as myocardial infarction and stroke. That is, diabetes mellitus, cardiodiabetes and metabolic syndrome are phenotypes of a common underlying pathophysiology.

Thus, the panel of biomarkers disclosed herein may find particular use for in diagnosing and treating disorders associated with cardiodiabetes.

The biomarkers and biomarker panels disclosed herein can be used in methods to diagnose, identify or screen subjects that have, do not have or are at risk for having disease; to monitor subjects that are undergoing therapies for disease; to determine or suggest a new therapy or a change in therapy; to differentially diagnose disease states associated with the disease from other diseases or within sub-classifications of disease; to evaluate the severity or changes in severity of disease in a subject; to stage a subject with the disease and to select or modify therapies or interventions for use in treating a subject with the disease. In an exemplary embodiment, the methods of the present invention are used to identify and/or diagnose subjects who are asymptomatic or presymptomatic for a disease. In this context, “asymptomatic” or “presymptomatic” means not exhibiting the traditional symptoms or enough abnormality for disease. In exemplary embodiments, the subject is normoglycemic.

In one aspect, the invention provides a method of determining a prognosis of a disease in a subject, diagnosing a disease in a subject, or treating a disease in a subject comprising taking a measurement of a biomarker panel in a sample from the subject.

The term “disease status” includes any distinguishable manifestation of the disease, including non-disease. For example, disease status includes, without limitation, the presence or absence of disease, the risk of developing disease, the stage of the disease, the progression of disease (e.g., progress of disease or remission of disease over time), the severity of disease and the effectiveness or response to treatment of disease.

As will be appreciated by those in the art, the biomarkers may be measured in using several techniques designed to achieve more predictable subject and analytical variability. On subject variability, many of the above biomarkers are commonly measured in a fasting state, commonly in the morning, providing a reduced level of subject variability due to both food consumption and metabolism and diurnal variation. All fasting and temporal-based sampling procedures using the biomarkers described herein may be useful for performing the invention. Pre-processing adjustments of biomarker results may also be intended to reduce this effect.

The term “sample” used herein refers to a specimen or culture obtained from a subject and includes liquids, gases and solids including, for example, tissue. In various exemplary embodiments, the sample comprises blood. A sample could be a fluid obtained from a subject including, for example, whole blood or a blood derivative (e.g. serum, plasma, or blood cells), ovarian cyst fluid, ascites, lymphatic, cerebrospinal or interstitial fluid, saliva, mucous, sputum, sweat, urine, or any other secretion, excretion, or other bodily fluids. As will be appreciated by those in the art, virtually any experimental manipulation or sample preparation steps may have been done on the sample. For example, wash steps may be applied to a sample. In one aspect, the invention provides a method of diagnosing a subject for a disease comprising taking a measurement of a biomarker panel in a sample from the subject; and correlating the measurement with the disease. The term “correlating” generally refers to determining a relationship between one type of data with another or with a state. In various embodiments, correlating the measurement with disease comprises comparing the measurement with a reference biomarker profile or some other reference value. In various embodiments, correlating the measurement with disease comprises determining whether the subject is currently in a state of disease.

The quantity or activity measurements of a biomarker panel can be compared to a reference value. Differences in the measurements of biomarkers in the subject sample compared to the reference value are then identified. In exemplary embodiments, the reference value is given by a risk category as described further below.

In various embodiments, the reference value is a baseline value. A baseline value is a composite sample of an effective amount of biomarkers from one or more subjects who do not have a disease, who are asymptomatic for a disease or who have a certain level of a disease. A baseline value can be the concentration of biomarkers measured in a sample obtained from a subject before a therapy is effected on the subject. A baseline value can also comprise the amounts of biomarkers in a sample derived from a subject who has shown an improvement in risk factors of a disease as a result of treatments or therapies. In these embodiments, to make comparisons to the subject-derived sample, the amounts of biomarkers are similarly calculated. A baseline value can also comprise the amounts of biomarkers derived from subjects who have a disease confirmed by an invasive or non-invasive technique, or are at high risk for developing a disease. Optionally, subjects identified as having a disease, or being at increased risk of developing a disease are chosen to receive a therapeutic regimen to slow the progression of a disease, or decrease or prevent the risk of developing a disease. A disease is considered to be progressive (or, alternatively, the treatment does not prevent progression) if the amount of biomarker changes over time relative to the reference value, whereas a disease is not progressive if the amount of biomarkers remains constant over time (relative to the reference population, or “constant” as used herein). The term “constant” as used in the context of the present invention is construed to include changes over time with respect to the reference value.

The biomarkers of the present invention can be used to generate a “reference biomarker profile” of those subjects who do not have a disease according to a certain threshold, are not at risk of having a disease or would not be expected to develop a disease. The biomarkers disclosed herein can also be used to generate a “subject biomarker profile” taken from subjects who have a disease or are at risk for having a disease. The subject biomarker profiles can be compared to a reference biomarker profile to diagnose or identify subjects at risk for developing a disease, to monitor the progression of disease, as well as the rate of progression of disease, and to monitor the effectiveness of disease treatment modalities. The reference and subject biomarker profiles of the present invention can be contained in a machine-readable medium, such as but not limited to, analog tapes like those readable by a VCR; optical media such as CD-ROM, DVD-ROM and the like; and solid state memory, among others.

The biomarker panels of the invention can be used by a practitioner to determine and effect appropriate therapies with respect to a subject given the disease status indicated by measurements of the biomarkers in a sample from the subject. Thus, in one aspect, the invention provides a method of treating a disease in a subject comprising taking a measurement of a biomarker panel in a sample from the subject, and effecting a therapy with respect to the subject. In one embodiment, the concentrations of the biomarkers of the biomarker panel increase or decrease according to the values described herein or stay the same in response to the therapy.

The terms “therapy” and “treatment” may be used interchangeably. In certain embodiments, the therapy can be selected from, without limitation, initiating therapy, continuing therapy, modifying therapy or ending therapy. A therapy also includes any prophylactic measures that may be taken to prevent disease.

In exemplary embodiments, effecting a therapy comprises administering a disease-modulating drug to a subject. Various examples of suitable disease-modulating drugs are described below. In exemplary embodiments, the disease-modulating drug is a DPPIV inhibitor or a GLP-1 analog. Generally, the drug can be a therapeutic or prophylactic used in subjects diagnosed or identified with a disease or at risk of having the disease. In certain embodiments, modifying a therapy refers to altering the duration, frequency or intensity of therapy, for example, altering dosage levels. In certain embodiments, a therapy comprises administering a combination of disease-modulating drugs (e.g., combinations including a DPPIV inhibitor or GLP-1 analog) to a subject. Any drug or combination of drugs described herein may be administered to a subject to treat a disease, such as cardiodiabetes.

In various embodiments, effecting a therapy comprises causing a subject to make or communicating to a subject the need to make a change in lifestyle, for example, increasing exercise, changing diet, reducing or eliminating smoking and so on. The therapy can also include surgery, for example, bariatric surgery. In various embodiments, effecting a therapy comprises causing a subject to follow or communicating to a subject the need to follow a dietary regimen having a high fiber and low carbohydrate content.

Measurement of biomarker concentrations allows for the course of treatment of a disease to be monitored. The effectiveness of a treatment regimen for a disease can be monitored by detecting one or more biomarkers of a biomarker panel in an effective amount from samples obtained from a subject over time and comparing the amount of biomarkers detected. For example, a first sample can be obtained prior to the subject receiving treatment and one or more subsequent samples are taken after or during treatment of the subject. Changes in biomarker concentrations across the samples may provide an indication as to the effectiveness of the therapy.

To identify therapeutics or drugs that are appropriate for a specific subject, a test sample from the subject can be exposed to a therapeutic agent or a drug, and the concentration of one or more biomarkers can be determined. Biomarker concentrations can be compared to a sample derived from the subject before and after treatment or exposure to a therapeutic agent or a drug, or can be compared to samples derived from one or more subjects who have shown improvements relative to a disease as a result of such treatment or exposure.

### Algorithms

In some embodiments, the correlation of biomarker measurements with drug response can comprise applying an algorithm of some kind to the results to determine drug response.

The term “formula,” “algorithm,” or “model” refers to any mathematical equation, algorithmic, analytical or programmed process, or statistical technique that takes one or more continuous or categorical inputs (herein called “parameters”) and calculates an output value, sometimes referred to as an “index” or “index value.” In various embodiments, the index is a drug response index. In various embodiments, a drug response index is based on measurements of a biomarker panel disclosed herein.

The algorithm may be as simple as determining whether or not the amount of each measured biomarker is above or below a respective level, threshold or cut-off. When multiple biomarkers are used, the algorithm may be, for example, a linear regression formula. Alternatively, the algorithm may be the product of any of a number of learning algorithms described herein or known in the art.

Non-limiting examples of formulas include sums, ratios, and regression operators, such as coefficients or exponents, biomarker value transformations and normalizations (including, without limitation, those normalization schemes based on clinical parameters, such as gender, age, or ethnicity), rules and guidelines, statistical classification models, and neural networks trained on historical populations. In one embodiment, linear and non-linear equations and statistical classification analyses are used to determine the relationship between levels of biomarkers detected in a subject sample and the subject's disease state. In panel and combination construction, of interest are structural and syntactic statistical classification algorithms, and methods of risk index construction, utilizing pattern recognition features, including established techniques such as cross-correlation, Principal Components Analysis (PCA), factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques, Shrunken Centroids (SC), StepAIC, Kth-Nearest Neighbor, Boosting, Decision Trees, Neural Networks, Bayesian Networks, Support Vector Machines, and Hidden Markov Models, among others. Other techniques may be used in survival and time to event hazard analysis, including Cox, Weibull, Kaplan-Meier and Greenwood models well known to those of skill in the art. Many of these techniques are useful either combined with a biomarker selection technique, such as forward selection, backwards selection, or stepwise selection, complete enumeration of all potential panels of a given size, genetic algorithms, or they may themselves include biomarker selection methodologies in their own technique. These may be coupled with information criteria, such as Akaike's Information Criterion (AIC) or Bayes Information Criterion (BIC), in order to quantify the tradeoff between additional biomarkers and model improvement, and to aid in minimizing overfit. The resulting predictive models may be validated in other studies, or cross-validated in the study they were originally trained in, using such techniques as Bootstrap, Leave-One-Out (LOO) and 10-Fold cross-validation (10-Fold CV). At various steps, false discovery rates may be estimated by value permutation according to techniques known in the art.

### Drug Treatments

In exemplary embodiments, effecting a therapy on a subject comprises administering a disease-modulating drug to the subject. The drug may be in any form suitable for administration to a subject, such forms including salts, prodrugs and solvates. The drug may be formulated in any manner suitable for administration to a subject, often according to various known formulations in the art or as disclosed or referenced herein. For example, the drug may be a component of a pharmaceutical composition comprising the drug and an excipient. Any drug, combination of drugs or formulation thereof disclosed herein may be administered to a subject to treat a disease.

The subject may be treated with one or more disease-modulating drugs until altered concentrations of the measured biomarkers return to a baseline value measured in a population not suffering from the disease, experiencing a less severe stage or form of a disease or showing improvements in disease biomarkers as a result of treatment with a disease-modulating drug. Additionally, improvements related to a changed concentration of a biomarker or clinical parameter may be the result of treatment with a disease-modulating drug and may include, for example, a reduction in body mass index (BMI), a reduction in total cholesterol concentrations, a reduction in LDL concentrations, an increase in HDL concentrations, a reduction in systolic and/or diastolic blood pressure, or combinations thereof.

A number of compounds such as a disease-modulating drug may be used to treat a subject and to monitor progress using the methods of the invention. In certain embodiments, the disease-modulating drug comprises a β-blocker, an angiotensin-converting enzyme (ACE) inhibitor, a diuretic, a calcium channel blocker, an angiotensin II receptor blocker, a antiplatelet agent, an anti-coagulant agent, a sulfonylurea (SU), a biguanide, an insulin, a glitazone (thiazolidinedione (TZD)), a nitrate, a non-steroidal anti-inflammatory agent, a statin, cilostazol, pentoxifylline, buflomedil or naftidrofuryl. In addition, any combination of these drugs may be administered.

The beneficial effects of these and other drugs can be visualized by assessment of clinical and laboratory biomarkers. For example, results from PROactive (Pfützner et al., Expert Review of Cardiovascular Therapy, 2006, 4: 445-459) and recent metanalyses have shown that these surrogate changes may translate into effective reduction of macrovascular risk in patients with type 2 diabetes mellitus.

In various embodiments, a glucagon-like peptide 1 (GLP-1) analog is administered to a subject. GLP-1 is secreted in response to food ingestion and plays a role in regulating postprandial blood glucose levels. Infusions of GLP-1 stimulate insulin secretion, inhibit glucagon secretion, lower blood glucose levels, increase β-cell mass and increase satiety. The use of GLP-1 as a drug is limited by the action of dipeptidyl peptidase-4 (DPPIV or DPP4), an enzyme that breaks down GLP-1. Accordingly, various GLP-1 analogs that are more resistant to degradation have been under development. GLP-1 analogs include derivatives of GLP-1 as well as alternative formulations of GLP-1 and GLP-1 derivatives. GLP-1 is an incretin, which is a class of hormones that increase the amount of insulin released from β-cells. Thus, an incretin, such as GLP-1 (particularly a GLP-1 analog) or gastric inhibitory peptide, as well as any derivative or formulation thereof, may find use in the various embodiments of the invention described herein. In some embodiments, a GLP-1 analog is a GLP-1 agonist, and these terms may be used interchangeably. In some embodiments, GLP-1 can be administered as a therapeutic to a subject, though it has a short half-life.

Numerous types and formulations of GLP-1 analogs are known in the art and find use in various embodiments disclosed herein. Examples of GLP-1 analogs include but are not limited to exenatide, liraglutide, albiglutide and taspoglutide. GLP-1 analogs have exemplary usefulness in treating various disorders, such as obesity. See Baggio and Drucker (posted Jul. 31, 2008) “Glucagon-like Peptide-1 Analogs Other Than Exentide”) Madsbad et al (2004) “Improved Glycemic Control with no Weight Increase in Patients with Type 2 Diabetes after Once-Daily Treatment with Long-Acting Glucagon-Like peptide 1 Analog Liraglutide (NN2211),” Emerging Treat. Tech. 27(6) 1335-1341). and U.S. Pat. No. 6,191,102, each incorporated by reference in their entirety.

In exemplary embodiments, a dipeptidyl peptidase IV (DPPIV) inhibitor is administered to a subject. DPPIV inhibitors are particularly useful in the various compositions and methods of the invention. A DPPIV inhibitor functions by decreasing the activity of DPPIV (also known as CD26), which is a proline-specific serine dipeptidyl aminopeptidase. DPPIV acts by cleaving two N-terminal residues, preferentially according to the formula X-Pro or X-Ala, from a number of polypeptides. In particular, DPPIV causes inactivation of GLP-1. One strategy in the treatment of type 2 diabetes is to target DPPIV using compounds that inhibit its activity. Through the use of DPPIV inhibitors, levels of active, circulating GLP-1 are increased, resulting in a prolongation of the hormone's effects, including reduced production of glucose through the inhibition of glucagon and increased insulin production.

Numerous types and formulations of DPPIV inhibitors are known in the art and find use in various embodiments disclosed herein. Examples of DPPIV inhibitors include but are not limited to alogliptin, linagliptin, saxagliptin, sitagliptin and vildagliptin.

In various embodiments, metformin is administered to a subject. Metformin (1,1-dimethylbiguanide or N,N-dimethylimidodicarbonimidic diamide) activates AMP-activated protein kinase (AMPK), a protein that plays an important role in cellular regulation of lipid and glucose metabolism. Activated AMPK reduces gluconeogenesis, at least in part by inhibition of gluconeogenic genes PEPCK and G6Pase. Metformin when administered to a subject is known to reduce hepatic glucose production and improve peripheral insulin sensitivity. Metformin has also been found to increase uptake of glucose in muscle and to reduce plasma triglyceride and nonesterified fatty acids. Metformin is a member of the biguanide class of compounds, and accordingly, any biguanide or biguanide formulation may be used in the various embodiments disclosed herein. Examples of biguanides other than metformin include phenformin and buformin. Derivatives of metformin may also find use in various embodiments.

In various embodiments, a sulfonylurea is administered to a subject. Sulfonylurea refers to a class of compounds that act by binding to ATP-sensitive potassium (K_{ATP}) channels of the β-cell, causing the channels to close. As a result, the β-cell membrane becomes depolarized, leading to an intracellular influx of Ca²⁺, which stimulates exocytosis of insulin-containing secretory granules. Sulfonylurea thus has been found to increase pancreatic release of insulin and can be referred to as an insulin secretogogue. Thus, various insulin secretogogues as known in the art may be used in the embodiments of the invention.

Numerous types and formulations of sulfonylureas are known in the art and find use in various embodiments disclosed herein. A number of sulfonylureas have been developed and classed into different generations. Examples of first generation sulfonylureas include acetohexamide, carbutamide, chlorpropamide, tolbutamide and tolazamide. Examples of second generation sulfonylureas include glipizide, gliclazide, glibenclamide (glyburide), gliquidone and glyclopyramide. Examples of third generation sulfonylureas include glimepiride. Any of these sulfonylureas and others known in the art, including any derivatives and formulations thereof may be used in the various embodiments of the invention.

In various embodiments, an insulin sensitizer is administered to a subject. An “insulin sensitizer” as used herein refers to any drug that enhances a subject's response to insulin. Exemplary insulin sensitizers act as agonists to PPAR, in particular to PPARγ. General classes of insulin sensitizers include, without limitation, glitazones (also referred to as thiazolidinediones (TZD)) and glitazars. In some embodiments, metformin is considered to be an insulin sensitizer. Numerous insulin sensitizers are known in the art and are useful in the present invention. Specific examples of insulin sensitizers include pioglitazone, rosiglitazone, netoglitazone (MCC-555), balaglitazone (DRF-2593), rivoglitazone (CS-011), troglitazone, MB-13.1258, 5-(2,4-dioxothiazolidin-5-ylmethyl)-2-methoxy-N-[4-(trifluoromethyl)benzyl]benzamide (KRP-297), FK-614, compounds described in WO/1999/058510 (e.g. (E)-4-[4-(5-methyl-2-phenyl-4-oxazolylmethoxy)benzyloxyimino]-4-phenylbutyric acid), aleglitazar, farglitazar (GI-262570), tesaglitazar (AZ-242), ragaglitazar (NN-622), muraglitazar (BMS-298585), reglitazar (JTT-501), ONO-5816, LM-4156, metaglidasen (MBX-102), naveglitazar (LY-519818), MX-6054, LY-510929, T-131, THR-0921 and the like. See WO/2005/041962 and US/2006/0280794.

In various embodiments, a glitazone is administered to a subject. In various embodiments, pioglitazone is administered to a subject. These and other drugs that are administered to treat a subject have been shown to affect concentrations of various biomarkers. In various embodiments, pioglitazone is administered with a statin, including but not limited to simvastatin. In various embodiments, pioglitazone may be administered with insulin or a GLP-1 analog, such as exenatide. In various embodiments, pioglitazone may be administered with an oral antidiabetic drug, including but not limited to a sulfonylurea (such as glimepiride), a biguanide (such as metformin), or a DPPIV-inhibitor (such as sitagliptin).

In various embodiments, a glinide is administered to a subject. Examples of glinides include but are not limited to repgalinide, nateglinide and mitiglinide.

In various embodiments, an α-glucosidase inhibitor is administered to a subject to treat a disease. An example of an α-glucosidase inhibitor is acarbose.

In various embodiments, an insulin is administered to a subject. The term “insulin” by itself refers to any naturally occurring form of insulin as well as any derivatives and analogs thereof. Different types of insulin may vary in the onset, peak occurrence and duration of their effects. Examples of insulin that may be useful in the present invention include but are not limited to regular human insulin, intermediate acting regular human insulin (e.g., NPH human insulin), Zn-retarded insulin, short acting insulin analog and long acting insulin analog. Examples of Zn-retarded insulin include but are not limited to lente and ultralente. Examples of short-acting insulin analog include but are not limited to lispro, aspart and glulisine. Examples of long-acting insulin analog include but are not limited to glargine and levemir.

Any drug or combination of drugs disclosed herein may be administered to a subject to treat a disease. The drugs herein can be formulated in any number of ways, often according to various known formulations in the art or as disclosed or referenced herein.

In various embodiments, one or more drug is combined with one or more treatment regimens such as diet, exercise and so on.

### Methods of Determining Treatment Efficacy

Additionally, therapeutic or prophylactic agents (i.e., drugs) suitable for administration to a particular subject can be identified by detecting one or more biomarkers in an effective amount from a sample obtained from a subject and exposing the subject-derived sample to a test compound that determines the amount of the one or more biomarker in the subject-derived sample. Accordingly, treatments or therapeutic regimens for use in subjects having a disease or subjects at risk for developing a disease can be selected based on the amounts of biomarkers in samples obtained from the subjects and compared to a reference value. Two or more treatments or therapeutic regimens can be evaluated in parallel to determine which treatment or therapeutic regimen would be the most efficacious for use in a subject to delay onset, or slow progression of a disease. In various embodiments, a recommendation is made on whether to initiate or continue treatment of a disease. Thus, the biomarker panels of the present invention can be used to determine the efficacy of treatment in a patient or subject.

Accordingly, in one aspect, the invention provides a method of assessing the efficacy of a first therapy on a subject comprising: taking a first measurement of a biomarker panel in a first sample from the subject; effecting the first therapy on the subject; taking a second measurement of the biomarker panel in a second sample from the subject; and making a comparison of the first measurement and the second measurement. In exemplary embodiments, the method comprises contacting a first or second sample from the subject with a solid support described herein. In some embodiments, the method further comprises effecting a second therapy on the subject based on the comparison. In exemplary embodiments, the first therapy comprises administering a DPPIV inhibitor or a GLP-1 analog to a subject. That is, in various embodiments, the method is for assessing the efficacy of a DPPIV inhibitor or a GLP-1 analog administered to a subject.

In some embodiments, a therapy comprises administering a disease-modulating drug to the subject. In these embodiments, changes in the levels of biomarkers between the first and second measurement allows a physician to either: a) keep the patient on a disease-modulating drug, as the changes in levels of certain biomarkers indicates the drug is working; b) keep the patient on the drug and adjust the dose; c) take the patient off the drug as efficacy is not present; and/or d) add an additional drug to the treatment, whether the patient is kept on the drug or not. Thus, effecting a second therapy in some embodiments comprises making a decision regarding the continued administration of the first disease-modulating drug.

In exemplary embodiments, the first therapy comprises administering a disease-modulating drug according to a first dosage regimen. In some embodiments, the first therapy comprises administering a combination of drugs according to a first dosage regimen. In exemplary embodiments, the combination comprises a DPPIV inhibitor or a GLP-1 analog. Thus, the methods of the invention can be used to test the efficacy of a combination of drugs, which can be modified for subsequent therapies according to differences in biomarker panel measurements.

A measurement of a biomarker panel will generally comprise the detection or observation of some characteristic (e.g., concentration (also referred to as a level)) of each member of the biomarker panel. A comparison of a first measurement and a second measurement will indicate a change, if any, in the measured characteristic for the biomarker of interest. A change as used herein may refer to any statistically relevant difference in the characteristic of a biomarker between a first measurement and a second measurement. A statistically relevant difference may be defined by the practitioner or by any art recognized method, and is generally defined herein. For example, a statistically relevant difference may be defined as a difference that surpasses a threshold defined by the practitioner. Thus, in various embodiments, making a comparison of the first measurement and the second measurement comprises determining the difference between the concentration of a biomarker in a first sample determined by the first measurement and the concentration of the biomarker in a second sample determined by the second measurement.

A change may refer to a single quantity, e.g., a 100% difference relative to a first measurement or may refer to a range, e.g., about 50% to about 100% difference or a ≧50% difference relative to a first measurement

A change may occur in either direction relative to a first measurement, i.e., the second measurement may be greater than or less than the first measurement. In some instances, there may be no change between measurements, and this absence of change may affect the therapeutic decision made by a practitioner in some embodiments.

Changes in the concentration of various combinations of biomarkers, such as those of a biomarker panel disclosed herein, will indicate to a practitioner a subject's responder status, i.e., whether or not a subject is a responder or nonresponder to a therapy. It should be appreciated that changes in biomarker concentrations can, in some cases, also indicate various degrees of response to a therapy. Thus, in some embodiments, a subject may be determined to be a strong responder, an intermediate responder or a weak responder. A subject associated with one of these response categories may optionally be given a different therapy compared to a subject associated with another. A practitioner can devise any number of response categories according to his or her needs.

Whether a subject is a responder or nonresponder to a therapy can be determined by the number and/or degree of changes observed in any combination of biomarkers of any biomarker panel disclosed herein. Identifying the responder status, which includes identifying nonresponder status, of a subject can aid the practitioner in choosing an appropriate therapy as discussed below.

One advantage of the biomarker panels of the invention is that they allow a practitioner to detect a response to a therapy, such as administration of a disease-modulating drug, within a short period of time, typically 1, 2, 3, 4, 5, 6 or 7 days, preferably within 1, 2, 3 or 4 days. Responder status can often be determined within 1 day after administration of the drug. Biomarker measurements made within 3 days after administration of the drug can be used to determine if changes in dosage are necessary. It may also be advantageous to detect a response to a therapy within 2, 3 or 4 weeks.

There are numerous ways of determining a subject's tendency to respond to a therapy. In various embodiments, a subject's responder status is based on a change observed for each biomarker of a biomarker panel or of a subset of the biomarker panel. In other words, if a biomarker panel comprises or consists of 9 biomarkers, a subject's responder status may be based on a change observed in 1, 2, 3, 4, 5, 6, 7, 8 or 9 biomarkers, in any combination.

In some embodiments, a change as defined above (e.g. an increase or a decrease, depending on the marker) in at least two markers (for example, selected from adiponectin, C-peptide, GLP-1, hsCRP, insulin and proinsulin) allows calling a patient a “responder”, e.g., someone to whom a drug is beneficial. In alternative embodiments, a change in at least 3, 4, 5, 6, 7, 8 or 9 of the markers allows the continuation of the drug.

### Response Tree

The direction and degree of changes in the biomarker levels used to indicate a response may vary depending on the type of response being detected. For example, when a DDPIV inhibitor is administered as a first therapy, one, a combination or all of the changes selected from an increase in adiponectin an increase in C-peptide concentration, an increase in GLP-1 concentration, an increase in insulin concentration and a decrease in proinsulin concentration occur(s) between a first sample and a second sample from a subject after a first therapy. In one embodiment, a subject is a responder to DPPIV inhibitor, when one, a combination or all of the changes selected from an increase in adiponectin, a decrease in C-peptide, a decrease in GLP-1, a decrease in insulin, and a decrease in proinsulin concentration occur(s) between a first sample and a second sample from a subject after a first therapy. In one embodiment, one, a combination or all of the changes selected from an increase in adiponectin concentration, a decrease in hsCRP concentration and a decrease in proinsulin concentration occur(s) between a first sample and a second sample from a subject after a first therapy. In one embodiment, one, a combination or all of the changes selected from an increase in adiponectin concentration, a decrease in hsCRP concentration, a decrease in proinsulin concentration, an increase in C-peptide concentration, an increase in GLP-1 concentration and an increase in insulin concentration occur(s) between a first sample and a second sample from a subject after a first therapy. In one embodiment, one, a combination or all of the changes selected from an increase or decrease in adiponectin concentration, an increase or decrease in C-peptide concentration, an increase or decrease in GLP-1 concentration, an increase or decrease in hsCRP concentration, an increase or decrease in insulin concentration and an increase or decrease in proinsulin concentration occur(s) between a first sample and a second sample from a subject after a first therapy.

When a GLP-1 analog is administered as a first therapy, one, a combination or all of the changes selected from an increase in C-peptide concentration, an increase in insulin concentration and a decrease in proinsulin concentration occur(s) between the first sample and the second sample from the subject after the first therapy. In one embodiment, a subject is a responder to DPPIV inhibitor, when one, a combination or all of the changes selected from an increase in adiponectin, a decrease in C-peptide, a decrease in insulin, and a decrease in proinsulin concentration occur(s) between a first sample and a second sample from a subject after a first therapy. In one embodiment, one, a combination or all of the changes selected from an increase in adiponectin concentration, a decrease in hsCRP concentration and a decrease in proinsulin concentration occur(s) between a first sample and a second sample from a subject after a first therapy. In one embodiment, one, a combination or all of the changes selected from an increase in adiponectin concentration, a decrease in hsCRP concentration, a decrease in proinsulin concentration, an increase in C-peptide concentration and an increase in insulin concentration occur(s) between a first sample and a second sample from a subject after a first therapy. In one embodiment, one, a combination or all of the changes selected from an increase or decrease in adiponectin concentration, an increase or decrease in C-peptide concentration, an increase or decrease in hsCRP concentration, an increase or decrease in insulin concentration and an increase or decrease in proinsulin concentration occur(s) between a first sample and a second sample from a subject after a first therapy.

As implied by the term “combination”, any of these changes may be included or excluded in a particular embodiment. In any of these embodiments, a biomarker change occurs according to a range disclosed herein. Other combinations of these changes (i.e. an increase or decrease beyond a reference value) and of changes in other panels could be used to determine a variety of responses.

In some embodiments, measurements of biomarker concentrations may be combined with genotyping of the subject to determine a therapy. That is, by combining biomarker concentrations with a subject's genotype for expressing, for example, a particular member of the CYP superfamily, a practitioner can choose a therapy or dosage accordingly. In an exemplary embodiment, a subject is genotyped for any CYP3A5 allele known in the art.

In various embodiments, CYP3A5 is used as a biomarker for determining a DPPIV inhibitor response; that is, a biomarker panel that includes adiponectin, C-peptide, GLP-1, hsCRP, insulin, proinsulin and CYP3A5.

It has been estimated that the members of the CYP3A family participate in the metabolism of about 50% of drugs known to undergo oxidative metabolism. The CYP3A family includes CYP3A4, which is the most abundant form, CYP3A5, CYP3A7 and CYP3A43. CYP3A5 is produced mainly in the liver and intestine and along with CYP3A4, accounts for the majority of catalytic activity in the CYP3A subfamily. CYP3A5 is detectable in a small percentage of adult whites and Asians, but has been found in 60% of a population of African Americans.

The genomic sequence of CYP3A5 can be found in RefSeq Accession Record NG_{—}007938.1.

In exemplary embodiments, the SNP status of a subject's genomic CYP3A5 gene is determined. In various embodiments, any allele of the CYP3A5 gene known in the art can be detected. See, for example, world wide web.cypalleles.ki.se/cyp3a5.htm.

Suitable capture probes for the detection and/or quantification of CYP3A5 include, but are not limited to, fragments of the complement of a nucleic acid strand corresponding to the sense or antisense sequence of the CYP3A5 gene. The capture probe can be complementary to either the sense or antisense strand of the gene. In general, as for all the capture probes outlined herein, the probes generally are between about 5 and about 100 basepairs in length, with about 6 to about 30, about 8 to about 28, and about 16 to about 26 being of particular use in some embodiments.

In one aspect, the invention provides a primer set comprising or consisting of one or more primers (e.g., one or more primer pairs) for amplifying a nucleic acid form of a biomarker for detection. In one embodiment, a primer set comprises or consists of primers for genotyping CYP3A5.

Once a practitioner has made a determination, based on the comparison of biomarker concentrations between a first and second measurement, as to whether a subject is a responder, nonresponder or a responder of a certain degree to a therapy (e.g. the administration of a disease-modulating drug), a practitioner may decide to effect a therapy based on this determination.

In some embodiments, a therapy comprises repeating or maintaining a previous therapy, such as administration of a disease-modulating drug. A practitioner might choose this therapy, if, for example, a subject that is administered a disease-modulating drug according to a first dosage regimen is determined to be a responder based on a change or set of changes described herein. In some embodiments, if the concentrations of all of the biomarkers of a biomarker panel that are expressed in the macrophage/monocyte decrease (e.g., MCP-1, MMP-9, NFκB, TNFα, IL6, p105, relA etc.), for example, at least 15% (or other appropriate value disclosed herein) compared to a first measurement, then the therapy comprises repeating or maintaining administration of a disease-modulating drug. In some embodiments, if the concentrations of all of the biomarkers of a biomarker panel decrease (except for biomarkers that tend to move in the opposite direction compared to others in indicating a response) or otherwise change to indicate a response as described herein compared to a first measurement, then the therapy comprises repeating or maintaining administration of a disease-modulating drug.

In some embodiments, a therapy comprises administering an additional drug to the subject, wherein the additional drug is different from a first administered drug. Other drugs useful in the present invention are described herein. An exemplary additional drug is a statin.

In some embodiments, a therapy comprises discontinuing a previous therapy, such as administration of a disease-modulating drug. A practitioner might choose this therapy, if, for example, a subject that is administered a disease-modulating drug according to a first dosage regimen is determined to be a nonresponder, e.g., there is no significant change in one or more of the biomarker concentrations. A practitioner might also choose this therapy, if, for example, a subject is a weak responder. For instance, a practitioner might determine that the risks of administering a drug outweighs the benefits of the weak response. In some embodiments, if the concentration of one or more biomarkers does not increase or decrease in a manner indicative of response to a first therapy (such as administration of a disease-modulating drug) as described herein, then a second therapy comprises discontinuing the first therapy.

In some embodiments, a therapy comprises administering a disease modulating drug according to a second dosage regimen. In these embodiments, the second dosage regimen will be different from the first dosage regimen associated with administration of the disease-modulating drug before measurement of a biomarker panel. In exemplary embodiments, the first dosage regimen comprises administering the disease modulating drug at a first dose and the therapy comprises administering the disease modulating drug at a second dose that depends on the degree of change in the expression of MCP-1 nucleic acid, MMP-9 nucleic acid or NFκB nucleic acid (or other nucleic acids of other panels), for example, or in the concentrations of some combination (such as all) of the biomarkers. In some embodiments, the therapy comprises administering a disease-modulating drug according to an adjusted dosage regimen compared to a previous dosage regimen.

The biomarkers of the invention show a statistically significant difference between different responses to a disease-modulating drug. In various embodiments, diagnostic tests that use these biomarkers alone or in combination show a sensitivity and specificity of at least about 85%, at least about 90%, at least about 95%, at least about 98% and about 100%.

The articles “a,” “an” and “the” as used herein do not exclude a plural number of the referent, unless context clearly dictates otherwise. The conjunction “or” is not mutually exclusive, unless context clearly dictates otherwise. The term “include” is used to refer to non-limiting examples.

All references, publications, patent applications, issued patents, accession records and databases cited herein, including in any appendices, are incorporated by reference in their entirety for all purposes.

1. A composition comprising a solid support comprising three four, five, or six biomarkers selected from the group comprising:
(a) a capture binding ligand selective for adiponectin,
(b) a capture binding ligand selective for C-peptide,
(c) a capture binding ligand selective for GLP-1,
(d) a capture binding ligand selective for hsCRP,
(e) a capture binding ligand selective for insulin, and
(f) a capture binding ligand selective for proinsulin.

2. The composition of claim 1, wherein one of the capture binding ligands comprises an antibody.

3. The composition of claim 1, wherein the composition further comprises three, four, five, or six biomarkers selected from the group comprising:
(a) a soluble binding ligand selective for adiponectin,
(b) a soluble binding ligand selective for C-peptide,
(c) a soluble binding ligand selective for GLP-1,
(d) a soluble binding ligand selective for hsCRP,
(e) a soluble binding ligand selective for insulin and
(f) a soluble binding ligand selective for proinsulin,
wherein each of the soluble capture ligands comprises a detectable label.

4. The composition of claim 3, wherein the detectable label is selected from the group consisting of a fluorophore or a conjugated enzyme, wherein the composition optionally further comprises a detector or a precipitating agent.

5-8. (canceled)

9. The use of the composition of claim 1 to determine a therapy for a subject experiencing diabetes or cardiodiabetes and optionally wherein the subject has been administered a DPPIV inhibitor.

10.-55. (canceled)

56. The use according to claim 9 comprising contacting the composition with a sample from the subject and measuring the concentrations of three or more biomarkers selected from the group consisting of adiponectin, C-peptide, GLP-1, hsCRP, insulin and proinsulin in the sample, thereby assaying the sample.

57. A method of assessing the efficacy of a first therapy on a subject comprising:
(a) contacting a first sample from the subject with the composition of claim 1;
(b) effecting the first therapy on the subject;
(c) contacting a second sample from the subject with the composition of claim 1; and
(d) making a comparison between the first and second measurements, wherein effecting the first therapy comprises administering a first disease-modulating drug to the subject according to a first dosage regimen; and
(e) effecting a second therapy on the subject based on the comparison;
wherein effecting the second therapy comprises making a decision regarding the continued administration of the first disease-modulating drug or comprises administering a second disease-modulating drug to the subject or administering the first disease-modulating drug according to an adjusted dosage regimen compared to the first dosage regimen.

58. The method of claim 57 wherein the adjusted dosage regimen depends on the degree of change in the concentration(s) of one, a combination or all of adiponectin, C-peptide, GLP-1, hsCRP, insulin and proinsulin between the first and second measurement.

59. The method of claim 57 wherein if one, a combination, or all of the changes selected from (a) an increase in C-peptide concentration, (b) an increase in GLP-1 concentration, (c) an increase in insulin concentration and (d) a decrease in proinsulin concentration occur(s) between the first and second measurements, then effecting the second therapy comprises repeating or maintaining the administration of the first disease-modulating drug.

60. The method of claim 57 wherein if one, a combination, or all of the changes selected from (a) an increase in C-peptide concentration by at least about 10%, (b) an increase in GLP-1 concentration by at least about 10%, (c) an increase in insulin concentration by at least about 10% and (d) a decrease in proinsulin concentration to below about 11 pmol/L occur(s) between the first and second measurements, then effecting the second therapy comprises repeating or maintaining the administration of the first disease-modulating drug.

61. The method of claim 57 wherein the first disease-modulating drug is selected from a group comprising DPPIV inhibitors, GLP-1 analogs, sulfonylureas, biguanide formulations, derivatives of biguanide formulations, insulin sensitizers, and α-glucosidase inhibitors.

62. The method of claim 57 wherein the second disease-modulating drug is selected from a group comprising DPPIV inhibitors, GLP-1 analogs, sulfonylureas, biguanide formulations, derivatives of biguanide formulations, insulin sensitizers, and α-glucosidase inhibitors.

63. The method of any of claim 57 wherein a sample comprises plasma or serum.

64. The method of claim 57 wherein the second sample is measured 1-3 days after affecting a first therapy.

65. A method for determining a drug response in a subject comprising
(a) taking a first and second measurement of a biomarker panel in a sample from the subject, the biomarker panel comprising or consisting of adiponectin, C-peptide, GLP-1, hsCRP, insulin and proinsulin using the composition claim 1, and
(b) correlating the measurement before and after administration of a disease modulating drug, wherein the modulating drug is selected from a group comprising DPPIV inhibitors, GLP-1 analogs, sulfonylureas, biguanide formulations, derivatives of biguanide formulations, insulin sensitizers, and α-glucosidase inhibitors.

66. The method of claim 65 wherein the drug is a DPPIV inhibitor and wherein the correlating step comprises calculating a DPPIV inhibitor response index.

67. The method of any of claim 65 wherein a sample comprises plasma or serum.

68. The method of claim 65 wherein the second sample is measured 1-3 days after affecting a first therapy.
